(19) United States
(12) Patent Application Publication
(10) Pub. No.: US 2024/0268808 A1
(43) Pub. Date: Aug. 15, 2024

(54) TISSUE PUNCTURE SEALING DEVICES AND METHODS

(71) Applicant: EDWARDS LIFESCIENCES CORPORATION, Irvine, CA (US)

(72) Inventors: Anne Bernadette Aragon Alcasid, Costa Mesa, CA (US); Yoon Hee Kwon, Mission Viejo, CA (US); Tiana Tran, Westminster, CA (US); Kokou Anani Amefia, Aliso Viejo, CA (US); Sydney Jade Fletcher, Huntington Beach, CA (US); James G. Wittel, Irvine, CA (US); Bianca Xochitl Aleman, Tustin, CA (US); Michael G. Valdez, Riverside, CA (US)

(21) Appl. No.: 18/647,256

(22) Filed: Apr. 26, 2024

Related U.S. Application Data
(63) Continuation of application No. PCT/US2022/047463, filed on Oct. 21, 2022.
(60) Provisional application No. 63/272,912, filed on Oct. 28, 2021.

(51) Int. Cl. A61B 17/00

(52) U.S. Cl. CPC A61B 17/0057; A61B 2017/00579; A61B 2017/0061; A61B 2017/00623; A61B 2017/00663

(57) ABSTRACT

Devices for puncturing, anchoring in the tissue of a human heart, and sealing the puncture and methods of puncturing, deploying anchors, and sealing the puncture are disclosed. The anchors are used to remodel the shape of the heart. The devices and methods use sealing structures to prevent blood leak through a passage formed by puncturing the heart tissue.

### RELATED APPLICATIONS

The present application is a continuation of Patent Cooperation Treaty application no. PCT/US2022/047463, filed on Oct. 21, 2022, which claims the benefit of U.S. Provisional Application No. 63/272,912, filed on Oct. 28, 2021, titled “TISSUE PUNCTURE SEALING DEVICES AND METHODS”, which are incorporated herein by reference in their entireties.

### BACKGROUND

Ischemic heart failure and systolic heart failure are conditions whereby the left ventricle becomes enlarged and dilated. With ischemic heart failure, a cardiac infarction occurs and the left ventricle remodels over a period of time, such as over days or months. With systolic heart failure, the left ventricle undergoes dilation for some other reason. For example, initial causes of systolic heart failure include chronic hypertension, mitral valve incompetency, and other dilated cardiomyopathies. A dilated heart, and particularly a dilated left ventricle, can significantly increase the tension and/or stress in the heart wall both during diastolic filling and systolic contraction, which contributes to ongoing dilatation of the left ventricular chamber.

Mitral valve incompetency or mitral valve regurgitation often accompanies ischemic and systolic heart failure. As the dilation of the ventricle proceeds, valve function may worsen. For example, as the dilation of the left ventricle progresses, the papillary muscles (to which the leaflets are connected via the chordae tendinea) may move radially outward and downward relative to the mitral valve, and relative to their normal positions. During this movement of the papillary muscles, however, the various chordae lengths remain substantially constant. This compromises the full closure ability of the leaflets by exerting tension prematurely on the leaflets. In addition, the enlargement of the left ventricle can cause the size of the mitral valve annulus to increase, while the area of the leaflets of the valve remains constant. This may lead to an area of less coaptation of the valve leaflets. Moreover, in normal hearts, the size of the mitral valve contracts during systole, aiding in valve coaptation. Right ventricular enlargement reduces annular contraction and distorts annular size, often exacerbating mitral valve regurgitation. The combination of the changes to the mitral valve annulus and the movement of the papillary muscles can result in a regurgitant mitral valve. This increase in regurgitation can, in turn, increase ventricular wall stress thereby advancing the dilation process, which can even further worsen mitral valve dysfunction.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Several devices for puncturing heart tissue, anchoring with the tissue of the heart, and sealing the puncture and methods of puncturing, deploying anchors, and sealing the puncture are disclosed. The anchors are used to remodel the shape of the heart. The devices and methods use sealing structures to prevent blood leak through a passage formed by puncturing the heart tissue.

In some implementations, a device for sealing a puncture in a human heart includes a hemostatic coil and at least one layer of a pliable material. The pliable material can extend coaxially around the hemostatic coil and can be configured to reduce an area between a suture line and an inner surface of a puncture channel.

In some implementations, the at least one layer of a pliable material forms a skirt having a first end and a second end. In some implementations, the first end of the skirt can form an opening that is smaller in size than an opening formed by the second end of the skirt. In some implementations, the skirt can be cinched at or near the first end of the skirt. In some implementations, the first end of the skirt can be offset from the distal end of the hemostatic coil. In some implementations, skirt has a tapered shape. In some implementations, the skirt has a variable width between the first end and the second end.

In some implementations, the at least one layer of a pliable material forms a skirt having plurality of petals joined to a collar. In some implementations, each of the plurality of petals has a tapered shape such that an end of each of the plurality of petals joined to the collar has a width that is less than a width of a free end of each of the plurality of petals.

In some implementations, a wrap extends along an outer surface of the hemostatic coil between the first end of the skirt to the distal end of the hemostatic coil.

In some implementations, the device includes one or more barbs configured to resist movement of the device toward an interior of the human heart when the device is positioned within the puncture channel. In some implementations, the one or more barbs extend from the hemostatic coil.

In some implementations, a device for sealing a puncture in a human heart includes a hemostatic coil and a fabric bead coupled to the hemostatic coil.

In some implementations, a method for sealing a puncture channel in a human heart having walls that at least partially define an internal chamber includes positioning a distal end of a delivery catheter adjacent an inner surface of a heart wall at a first location and delivering a hemostatic plug comprising a hemostatic coil and at least one layer of a pliable material through the catheter to the puncture channel such that the hemostatic plug is positioned within the puncture channel and reduces a flow of blood through the puncture channel. In some implementations, the pliable material can extend coaxially around the hemostatic coil

In some implementations, the at least one layer of pliable material forms a skirt having a first end and a second end, wherein the first end of the skirt forms an opening that is smaller in size than an opening formed by the second end of the skirt. In some implementations, the skirt is cinched at or near the first end of the skirt. In some implementations, the first end of the skirt is offset from the distal end of the hemostatic coil.

In some implementations, the skirt has a waist configured to enable the skirt to expand laterally when the skirt is compressed in a longitudinal direction extending from the first end to the second end. In some implementations, the skirt comprises a plurality of petals joined to a collar.

In some implementations, the method includes delivering a tissue anchor through the catheter to the heart wall such that the tissue anchor anchors to the heart wall. In some implementations, the tissue anchor includes a first suture line attached and extends into the internal chamber. In some implementations, delivering the tissue anchor is performed prior to the step of delivering the hemostatic plug. In some implementations, delivering the tissue anchor is performed simultaneously with the step of delivering the hemostatic plug. In some implementations, the hemostatic plug is coupled to the tissue anchor through the first suture line.

The above method(s) can be performed on a living animal or on a simulation, such as on a cadaver, cadaver heart, anthropomorphic ghost, simulator (e.g., with body parts, heart, tissue, etc. being simulated).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a diastolic phase, in which the chordae tendineae are shown attaching the leaflets of the mitral and tricuspid valves to ventricle walls;

FIG. 4 illustrates a healthy mitral valve with the leaflets closed as viewed from an atrial side of the mitral valve;

FIG. 5 illustrates a dysfunctional mitral valve with a visible gap between the leaflets as viewed from an atrial side of the mitral valve;

FIG. 6 illustrates a cutaway view of the human heart showing the papillary muscles;

FIG. 7 illustrates a cutaway view of the human heart showing the multilayer heart wall;

FIG. 8 is an enlarged cutaway view of the human heart wall;

FIG. 9 is an enlarged cutaway view of the human heart wall of FIG. 8 showing a needle about to pierce the heart wall;

FIG. 10 is an enlarged cutaway view of the human heart wall of FIG. 8 showing a needle inserted into the myocardium of the heart wall;

FIG. 11 is an enlarged cutaway view of the human heart wall of FIG. 8 showing a needle inserted into the parietal tissue of the pericardium of the heart wall;

FIG. 12 is an enlarged cutaway view of the human heart wall of FIG. 8 showing a needle inserted into the pericardial cavity of the heart wall;

FIG. 13 is an enlarged cutaway view of the human heart wall of FIG. 8 showing a needle injecting a dye into the pericardial cavity of the heart wall;

FIG. 14 is an enlarged cutaway view of the human heart wall of FIG. 8 showing a needle inserted into the pericardial cavity of the heart wall and a catheter adjacent the endocardium of the heart wall;

FIG. 15 is an enlarged cutaway view of the human heart wall of FIG. 8 showing a needle and a catheter inserted into the pericardial cavity of the heart wall;

FIG. 16 is an enlarged cutaway view of the human heart wall of FIG. 8 showing an anchor deployed into the pericardial cavity of the heart wall;

FIG. 17 is a view similar to the view of FIG. 16 where the anchor is deployed through an introducer or needle;

FIG. 18 is a view similar to FIG. 16 where the anchor is deployed over an introducer or needle;

FIG. 19 is another view similar to FIG. 16 where the anchor is deployed over an introducer or needle;

FIG. 20 is an enlarged cutaway view of the human heart wall of FIG. 8 showing the anchor of FIG. 16 seated in the pericardial cavity of the heart wall;

FIG. 21 is an enlarged cutaway view of the human heart wall of FIG. 8 showing the needle extending through the heart wall;

FIG. 22 is an enlarged cutaway view of the human heart wall of FIG. 8 showing the needle extending through the heart wall and a catheter adjacent the endocardium of the heart wall;

FIG. 23 is an enlarged cutaway view of the human heart wall of FIG. 8 showing the needle and a catheter extending through the heart wall;

FIG. 24 is an enlarged cutaway view of the human heart wall of FIG. 8 showing an anchor deployed through the catheter;

FIG. 25 is an enlarged cutaway view of the human heart wall of FIG. 8 showing the anchor seated against the parietal tissue of the heart wall;

FIG. 26 illustrates a cutaway view of the human heart showing a needle inserted through a papillary muscle of the heart and through the heart wall;

FIG. 27 illustrates a cutaway view of the human heart of FIG. 26 showing the needle inserted through the papillary muscle and a delivery catheter positioned adjacent the papillary muscle;

FIG. 28 is an enlarged cutaway view of the human heart wall showing a needle and a catheter inserted through a papillary muscle of the heart and into the pericardial cavity of the heart wall;

FIG. 29 is an enlarged cutaway view of the human heart wall of FIG. 28 showing an anchor deployed into the pericardial cavity of the heart wall;

FIG. 30 is an enlarged cutaway view of the human heart wall of FIG. 28 showing an anchor seated in the pericardial cavity of the heart wall;

FIG. 31 illustrates a cutaway view of the human heart showing an anchor seated against the exterior of the heart wall with a line extending through a papillary muscle;

FIG. 32 illustrates a cutaway view of the human heart showing a first anchor seated against the exterior of the heart wall with a line extending through a first papillary muscle and a second anchor seated against the exterior of the heart wall with a second line extending through a second papillary muscle;

FIG. 33 illustrates the cutaway view of the human heart of FIG. 32 showing the first line and a second line being pulled through a connector to pull the papillary muscles toward one another;

FIG. 34 shows the first and second lines secured in the connector and trimmed;

FIG. 35 illustrates the cutaway view of the human heart of FIG. 34 showing a third anchor seated against the interventricular septum and a third line coupled to the first and second lines;

FIG. 36 illustrates a cutaway view of the human heart showing a first anchor seated against the exterior of the heart wall with a first line extending through a papillary muscle and a second anchor seated against the interventricular septum and a second line connected to the first line;

FIG. 37 illustrates a cutaway view of the human heart showing a first anchor seated against the exterior of the heart wall with a first line extending through the heart wall and a second anchor seated against the interventricular septum and a second line connected to the first line;

FIG. 38 illustrates an example of an anchor for a papillary muscle approximation system, the anchor illustrated in an extended configuration;

FIG. 39 illustrates the anchor of FIG. 38 in a non-deployed, extended configuration;

FIG. 40 illustrates the anchor of FIG. 38 in a deployed configuration;

FIG. 41 illustrates the anchor of FIG. 38 in a non-deployed, extended configuration along with a hemostatic plug;

FIG. 42 illustrates the anchor of FIG. 38 in a deployed configuration along with a hemostatic plug;

FIG. 43 illustrates a delivery sheath and a steerable catheter for delivering the anchor of FIG. 38;

FIG. 44 illustrates an anchoring delivery catheter extending from the delivery sheath and the steerable catheter of FIG. 43;

FIG. 45 illustrates a needle extending from the anchoring delivery catheter of FIG. 44;

FIG. 46 illustrates the anchor extending along the needle of FIG. 45;

FIG. 47 illustrates the anchor and hemostatic plug along with the needle and a pusher of the delivery system of FIG. 42;

FIG. 48 illustrates the anchor and hemostatic plug of FIG. 47;

FIG. 49 illustrates the anchor and hemostatic plug of FIG. 47 with lines attached;

FIG. 50 illustrates the anchor with lines attached without a hemostatic plug;

FIG. 51 illustrates the delivery system deploying the anchor and hemostatic plug without lines;

FIG. 52 illustrates the delivery system, anchor, and hemostatic plug of FIG. 51 with lines;

FIG. 53 illustrates the delivery system, anchor, and hemostatic plug of FIG. 52 with the anchor in a deployed state;

FIG. 54 illustrates an enlarged view of the anchor and hemostatic plug in a deployed state;

FIG. 55 illustrates the anchor and hemostatic plug in a deployed state with the needle and delivery catheter of the delivery system withdrawn;

FIG. 56 is an enlarged cutaway view of the human heart wall showing the delivery sheath and steerable catheter positioned adjacent the heart wall;

FIG. 57 is an enlarged cutaway view of the human heart wall showing the delivery catheter anchored to the myocardium of the heart wall;

FIG. 58 is an enlarged cutaway view of the human heart wall showing the needle inserted into the pericardial cavity of the heart wall;

FIG. 59 is an enlarged cutaway view of the human heart wall showing a needle injecting a dye into the pericardial cavity of the heart wall;

FIG. 60 is an enlarged cutaway view of the human heart wall showing the anchor being deployed along the needle;

FIG. 61 is an enlarged cutaway view of the human heart wall showing the anchor extending inside the pericardial cavity;

FIG. 62 is an enlarged cutaway view of the human heart wall showing the anchor deployed inside the pericardial cavity;

FIG. 63 is an enlarged cutaway view of the human heart wall showing the anchor being seated in the pericardial cavity;

FIG. 64 is an enlarged cutaway view of the human heart wall showing the anchor seated in the pericardial cavity and the delivery system removed;

FIG. 65 illustrates an example of an anchor having a hemostatic plug in the form of a skirt;

FIG. 66 illustrates an example of an anchor having a hemostatic plug in the form of a skirt cinched at one end;

FIG. 67 illustrates an example of an anchor having a hemostatic plug in the form of a skirt formed from a weave of nitinol and PET;

FIG. 68 illustrates an example of an anchor having a hemostatic plug in the form of a skirt having a wrap thereon;

FIG. 69 illustrates another example of an anchor having a hemostatic plug in the form of a skirt having a wrap thereon;

FIG. 70 illustrates an example of an anchor in elongated form having a hemostatic plug in the form of skirt over a sleeve;

FIG. 71 illustrates the anchor of FIG. 70 in a deployed configuration;

FIG. 72 illustrates an example of an anchor in elongated form having a hemostatic plug in the form of hemostatic coil with a fabric bead;

FIG. 73 illustrates the anchor of FIG. 72 in a deployed configuration;

FIG. 74 illustrates an example of an anchor having a hemostatic plug in the form of a skirt having a waist;

FIG. 75 illustrates the skirt of FIG. 74 in a compressed configuration;

FIG. 76 illustrates an example of a hemostatic plug in the form of a skirt having a plurality of petals;

FIG. 77 illustrates various configurations for the plurality of petals;

FIG. 78 illustrates an example of an anchor having a hemostatic plug in the form of a skirt having a plurality of petals;

FIG. 79A is a top down view of an example of an anchor having a hemostatic plug in the form of a plurality of petals formed from a hemostatic tube in a closed configuration;

FIG. 79B is a side view of the example of the anchor of FIG. 79A in an open configuration;

FIG. 80A illustrates an example of a hemostatic plug having a plurality of barbs and a fabric bead;

FIG. 80B illustrates an example of a hemostatic plug having a plurality of barbs and a plurality of petals;

FIG. 80C illustrates an example of a hemostatic plug having a plurality of barbs and a skirt;

FIG. 81 illustrates an example of a hemostatic plug having a plurality of barbs and a collapsible feature; and

FIG. 82 illustrates the hemostatic plug of FIG. 81 in a puncture channel.

### DETAILED DESCRIPTION

The following description refers to the accompanying drawings, which illustrate implementations of the present disclosure. Other implementations having different structures and operation do not depart from the scope of the present disclosure.

Example implementations of the present disclosure are directed to systems, devices, methods, etc. for repairing a defective heart valve. For example, various implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques and methods herein can be performed on a living animal or on a simulation, such as on a cadaver, cadaver heart, simulator (e.g., with the body parts, heart, tissue, etc. being simulated), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).”

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

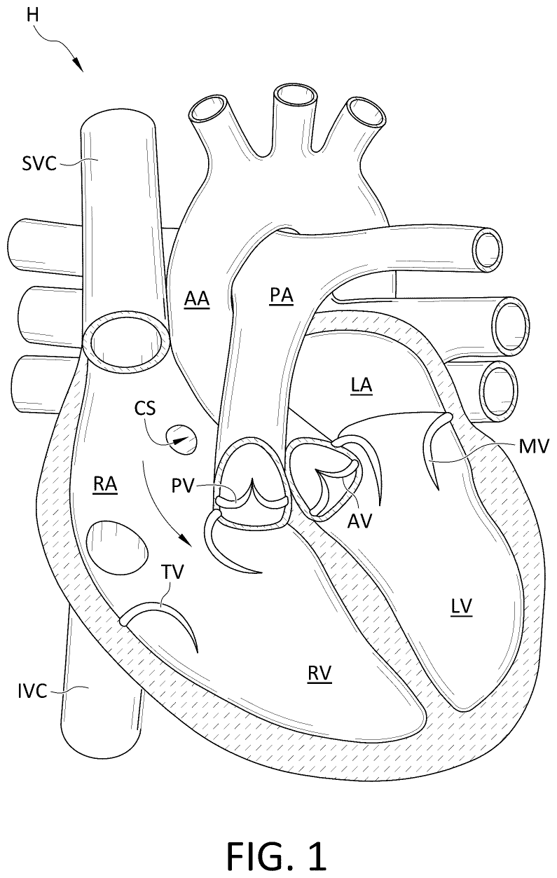

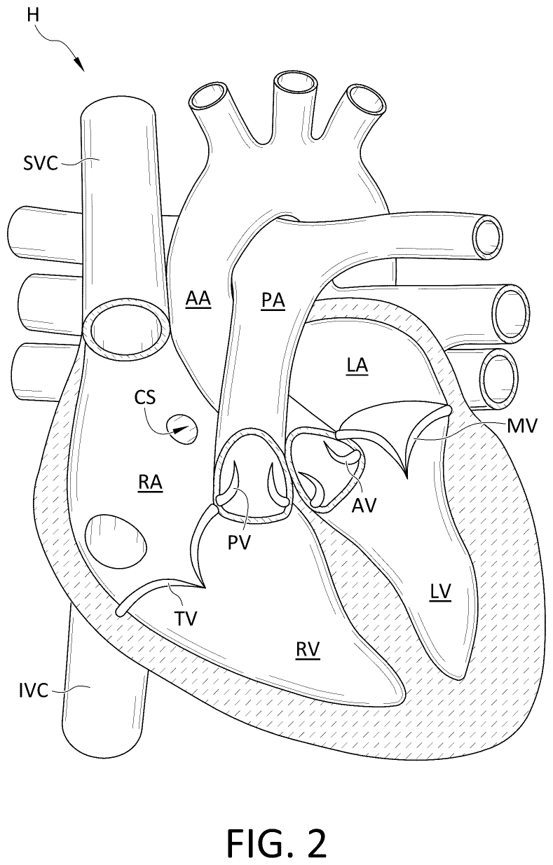

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets extending inward across the respective orifices that come together or “coapt” in the flow stream to form the one-way, fluid-occluding surfaces. The remodeling devices, systems, and methods of the present application are described primarily with respect to the left ventricle LV. Therefore, anatomical structures of the left side of the heart will be explained in greater detail. It should be understood that the devices, systems, and methods described herein may also be used in remodeling the right ventricle.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA, and blood is collected in the left atrium from the pulmonary vein.

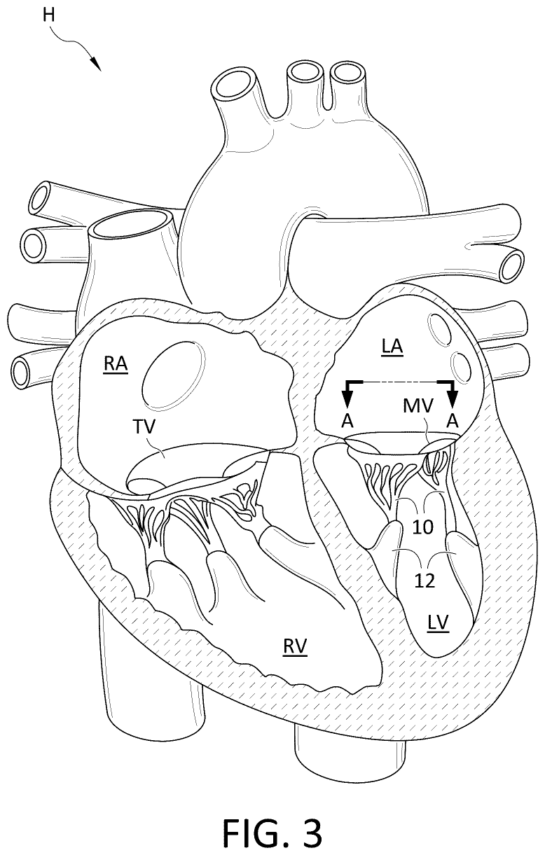

Referring now to FIGS. 1-5, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIG. 3, the mitral valve MV is anchored to the wall of the left ventricle LV by chordae tendineae 10. The chordae tendineae 10 are cord-like tendons that connect the papillary muscles 12 (i.e., the muscles located at the base of the chordae tendineae and within the walls of the left ventricle) to the leaflets 20, 22 of the mitral valve MV. The papillary muscles 12 serve to limit the movements of the mitral valve MV and prevent the mitral valve from being inverted or prolapsed. The mitral valve MV opens and closes in response to pressure changes in the left atrium LA and the left ventricle LV. The papillary muscles do not open or close the mitral valve MV. Rather, the papillary muscles brace the mitral valve MV against the high pressure needed to circulate blood throughout the body. Together the papillary muscles and the chordae tendineae are known as the subvalvular apparatus, which functions to keep the mitral valve MV from prolapsing into the left atrium LA when the mitral valve closes.

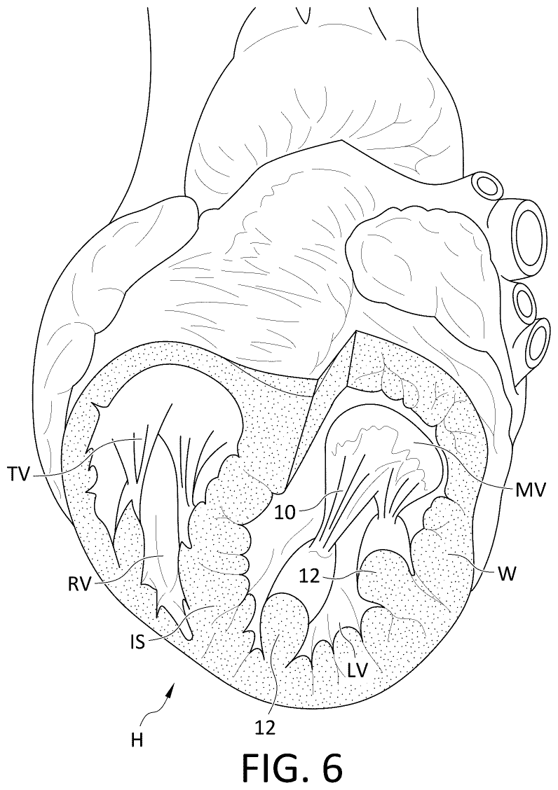

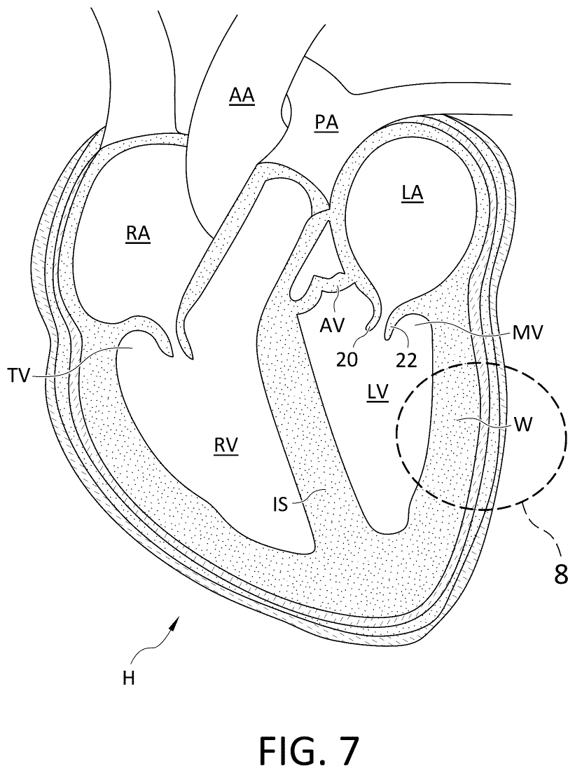

FIG. 6 is a cutaway view of the human heart with the section through the papillary muscles of the left ventricle. The right ventricle RV is separated from the left ventricle LV by the interventricular septum IS. The mitral valve leaflets 20, 22 (shown FIG. 7) extend inward across the respective orifices and come together or “coapt” in the flowstream to form the one-way, fluid-occluding surfaces. The devices and methods for remodeling the shape of the heart walls W are described primarily with respect to the left ventricle LV. The devices and methods can be used to approximate the papillary muscles in some example ts, which are also described primarily with respect to the left ventricle LV. In addition to reducing the size of the ventricle to increase the ventricular function, bringing the papillary muscles closer together can cause the valve leaflets to coapt and prevent mitral valve regurgitation. It should be understood that the devices described herein may also be used in remodeling the right ventricle RV and approximate the papillary muscles of the tricuspid valve TV.

In some implementations, the devices described by the present application are used to remodel the shape of a ventricle to improve heart function. Heart function can be improved by reducing the size of the ventricle, approximating the papillary muscles, and/or correcting the function of the mitral valve MV. In one example, the devices are configured to reshape the wall of a human heart H in a way that causes the mitral valve MV to prevent blood from regurgitating from the left ventricle LV and back into the left atrium LA.

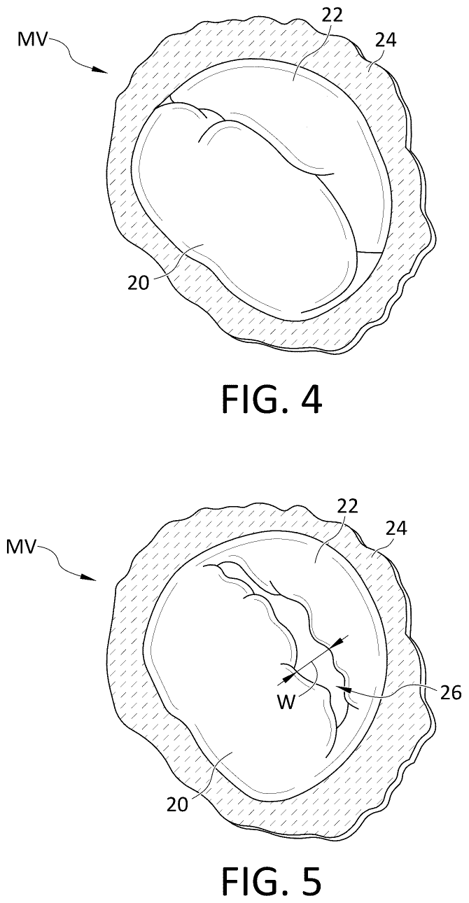

When a healthy mitral valve MV is in a closed position, the leaflets 20, 22 coapt, as shown in FIG. 4, which prevents blood from leaking from the left ventricle LV to the left atrium LA. Regurgitation can occur when one or both of the leaflets 20, 22 of the mitral valve MV prolapse into the left atrium LA during systole or the leaflets fail to coapt or close together against one another. This prolapse and/or a failure to coapt causes a gap 26 between the leaflets 20, 22, which allows blood to flow back into the left atrium LA from the left ventricle LV during systole.

The devices and procedures disclosed herein make reference to remodeling the left ventricle, with the possible consequence of better coaption of the leaflets of the mitral valve. However, it should be understood that the devices and concepts provided herein can be used to remodel the right ventricle, with the possible consequence of better coaption of the tricuspid valve TV leaflets.

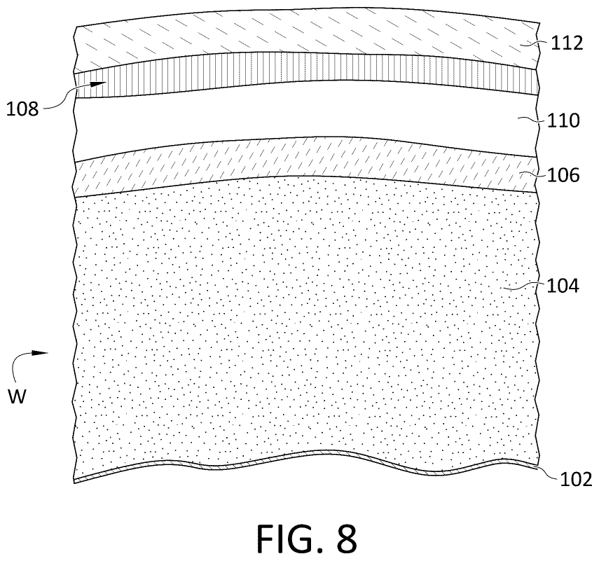

Referring now to FIG. 8, an enlarged cutaway view of the human heart wall W of FIG. 7 is illustrated. The heart wall W has multiple layers, which include the endocardium 102, the myocardium 104, and the epicardium 106. The endocardium 102 is the most inner layer of the heart H. It forms the inner layer of all four heart chambers and is directly connected to all the inner cardiac appendages, such as the bicuspid valve BV, the tricuspid valve TV, the pulmonary valve (not shown), the aortic valve AV, and the chordae tendineae CT by way of the papillary muscles 12.

The myocardium 104 sits between the inner endocardium 102 and the outer epicardium 106. The myocardium 104 is the basic muscle that makes up the heart H and it functions by providing a scaffolding for the heart chambers. The myocardium 104 contracts and relaxes the cardiac walls so that blood can pass between the chambers.

The epicardium 106 is a visceral layer of serous pericardium. The epicardium is the innermost of the two layers of the pericardium. The epicardium covers the external surfaces of the heart. It is directly fused with the myocardium internally. It is comprised mainly of connective tissue and protectively encompasses the heart.

The pericardium 108 is the double-walled sac that contains the heart and roots of the great vessels that leave from or enter the heart. A space is formed between epicardium 106 and the serous layer of the pericardium 108, which is known as the pericardial cavity 110, which contains pericardial fluid. A layer of parietal pericardium 112 is disposed around the heart. The outer parietal layer (i.e., the parietal pericardium 112) and the inner serous pericardium layer (i.e., the pericardium 108) are on the outside of the pericardial cavity 110.

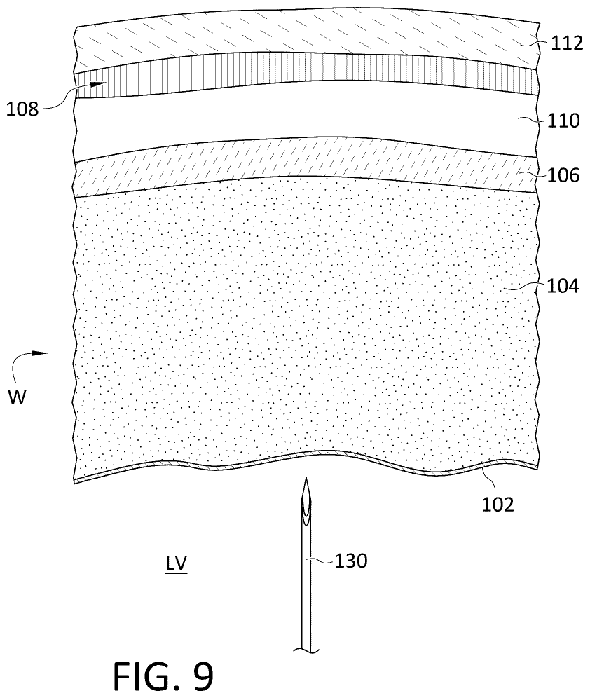

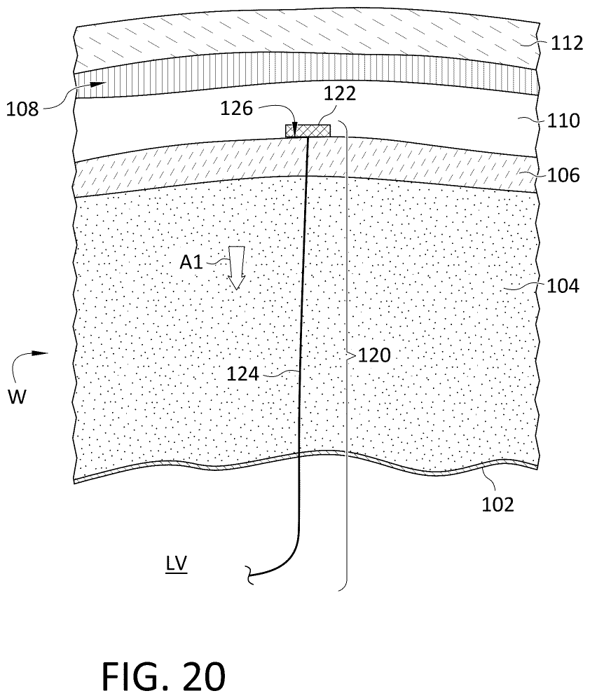

Referring to FIGS. 9-20, an example of a device 120 for remodeling the shape of a heart wall W, and an example of a system and method for delivering and deploying the device 120 into the pericardial cavity 110 is illustrated. Referring to FIG. 20, the device 120 includes an anchor 122 and a line 124 engaging or connected to the anchor 122 and extending therefrom. The line 124 can take a wide variety of different forms. Examples of lines 124 include, but are not limited to, sutures, wires, cables, chords, bendable rods, any combination thereof, etc. The line 124 can be any element or combination of elements that is configured to extend from the anchor 122, through the heart wall W, and into the internal chamber.

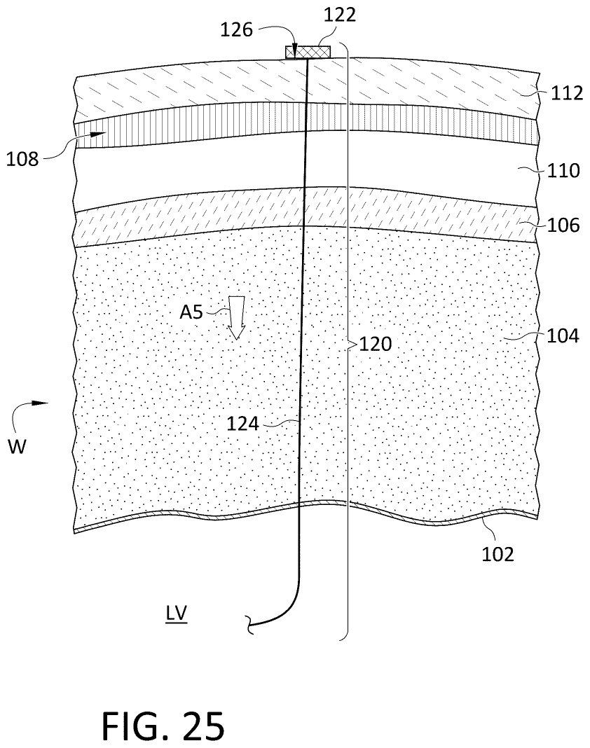

The anchor 122 is configured to be positioned against a surface 126 facing outward relative to an internal chamber of the heart H, such as for example, the left ventricle LV or right ventricle of the heart H. In the illustrated example of FIG. 20, the outward facing surface 126 is a portion of the epicardium 106 and the anchor 122 is disposed in the pericardial cavity 110. In the example illustrated by FIG. 25, the outward facing surface 126 is the pericardium 108.

The anchor 122 can be configured in a variety of ways. Any configuration that can be positioned to engage an outward facing surface 126 of the heart wall W to support pulling a portion of the heart wall W inward (i.e., toward the internal chamber) can be used. For example, the anchor 122 can be a pledget, a sufficiently sized knot formed in the line 124, a stop, or some other line anchoring device. The anchor 122 can be collapsible/expandable or reconfigurable, such that the anchor can be delivered through a catheter or sheath in a delivered state (e.g., collapsed or elongated) that fits within a lumen of the catheter, and can be reshaped or expanded to a deployed state once it has been delivered to the appropriate location. In one example, the anchor 122 includes a shape-memory alloy—such as Nitinol—to provide shape-setting capability.

Referring to FIG. 9, in one example, deployment of the device 120 (not shown in FIG. 9) includes delivering a piercing device 130 into an internal chamber (e.g., left ventricle LV) of the heart H and adjacent the heart wall W. The piercing wall W, such as for example, a needle, wire, or other similar device. In the illustrated implementation, the piercing device 130 is a needle or hollow wire having an inner passage (not shown) and an opening 131 proximate the distal end 133 of the piercing device the fluidly connects the inner passage (not shown) to the exterior of the piercing device 130. However, in other implementations, the piercing device is not hollow. In the illustrated implementation, the piercing device 130 is pointed or has a sharp tip. However, in other implementations, the piercing device 130 has a blunt tip.

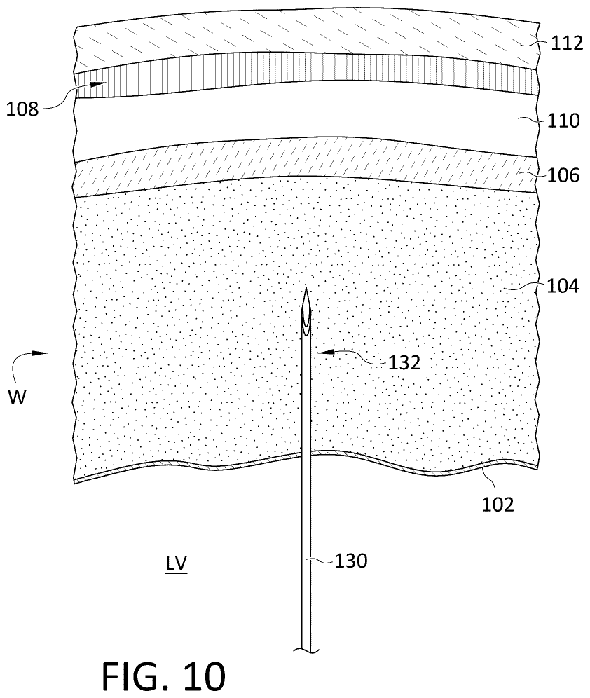

In FIG. 10, the piercing device 130 is extended into the heart wall W through the endocardium 102 and into the myocardium 104 to create a passage 132 through the heart wall W. The piercing device 130, however, is not yet sufficiently inserted to deploy the anchor 122 into the pericardial cavity 110. Thus, the piercing device 130 in FIG. 10 is shown in an under-inserted position.

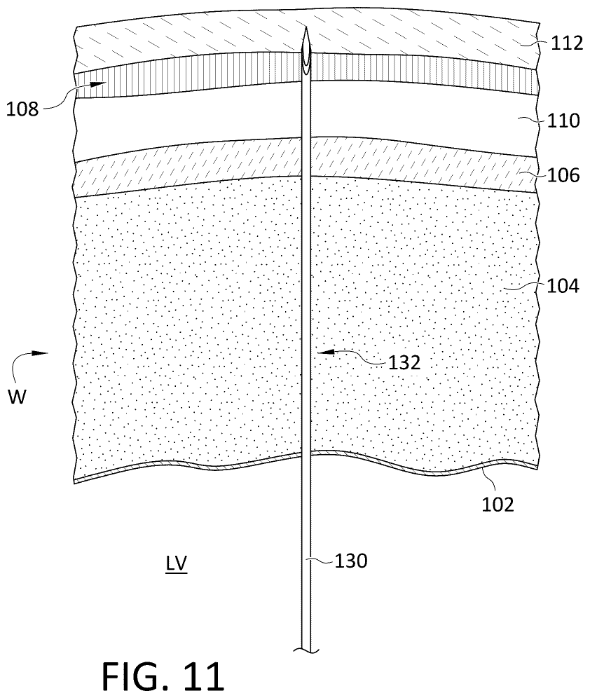

In FIG. 11, the piercing device 130 is extended through the endocardium 102, the myocardium 104, the epicardium 106, and the pericardium 108, and into the external parietal pericardium tissue 112 to extend the passage 132 in the heart wall W. The piercing device 130, however, has extended past the pericardial cavity 110 where the anchor 122 is to be positioned in this example. Thus, the piercing device 130 in FIG. 11 is shown in an over-inserted position for this implementation (however, this can be the correct position for other implementations).

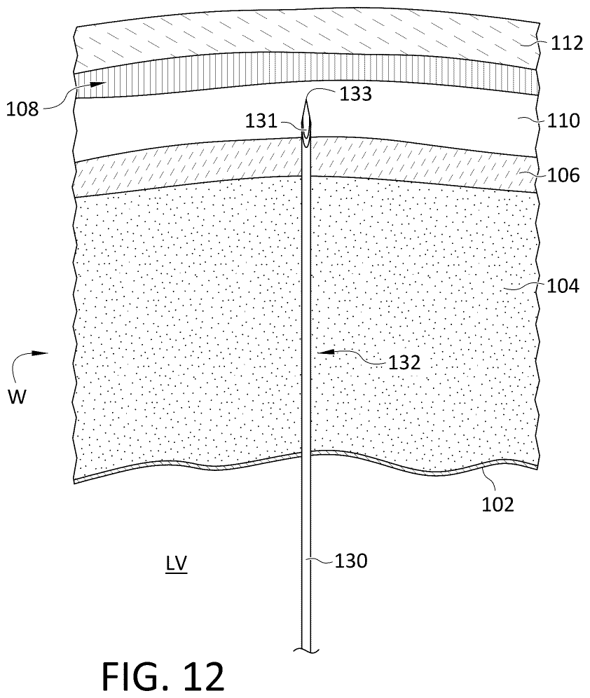

In FIG. 12, the piercing device 130 is extended through the endocardium 102, the myocardium 104, the epicardium 106, and into the pericardial cavity 110, such that the opening 131 and the distal end 133 are within the within the pericardial cavity 110. Thus, the piercing device 130 in FIG. 12 is properly positioned for deploying the anchor 122 into the pericardial cavity 110.

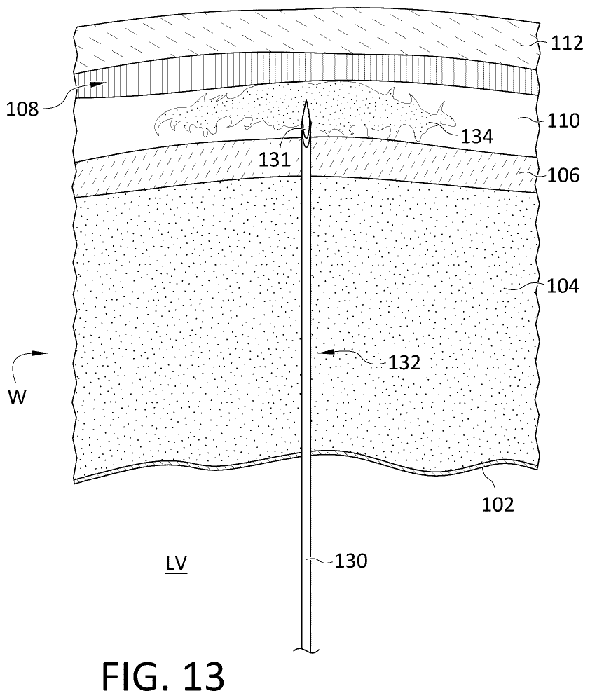

In some implementations, proper positioning of the piercing device 130 is optionally verified. The proper positioning of the piercing device 130 can be verified in a wide variety of different ways. For example, the positioning of the piercing device can be visually determined by providing the piercing device with a marker, such as a radiopaque marker, by discharging a material into cavity, such as the pericardial cavity, by sensing a pressure required to discharge fluid from the piercing device, by sensing a force required to advance the piercing device, by positioning a small guide wire in the pericardial cavity, and/or with electrical signals, such as by electrical signals provided by and/or sensed by the piercing device, etc. In some implementations, to verify that the piercing device 130 is properly positioned for deploying the anchor 122 into the pericardial cavity 110, a dye 134, or other detectable fluid, is delivered through the piercing device 130 and injected into the pericardial cavity 110, as shown in FIG. 13. The dye 134 can be detected by any suitable technique such as X-ray or other imaging techniques, to verify that the piercing device 130 is properly positioned.

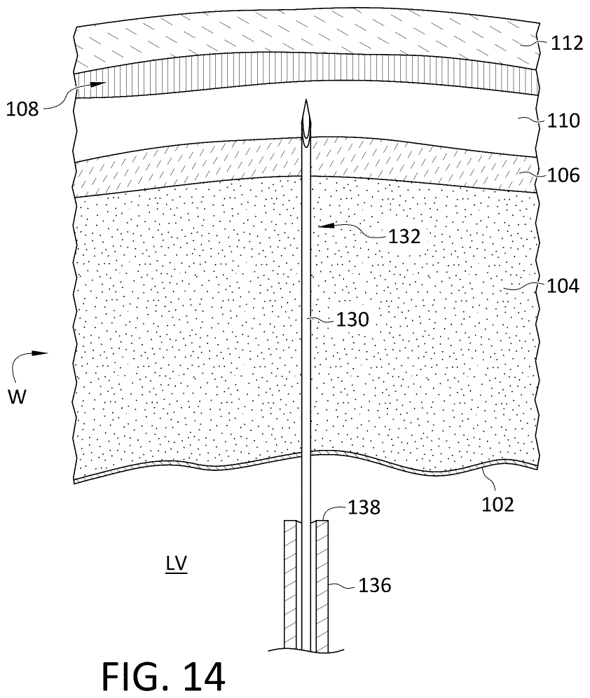

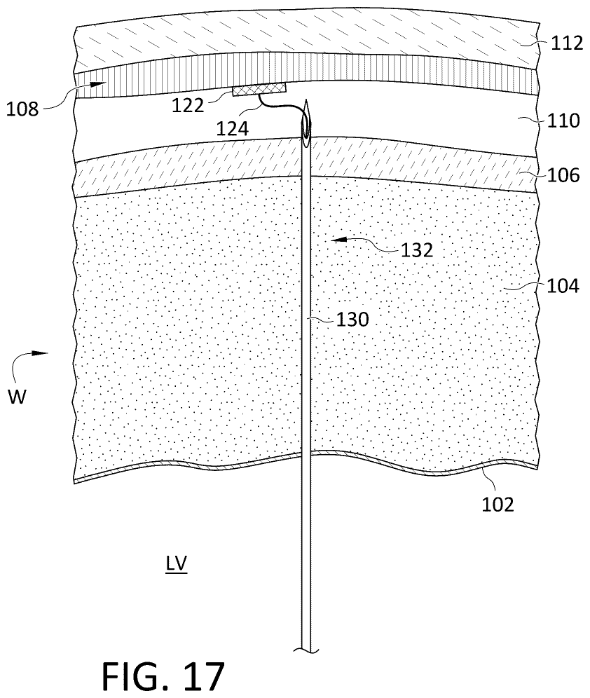

In FIG. 14, the piercing device 130 is extended into the pericardial cavity 110 and a delivery catheter 136 is positioned within the heart chamber (e.g., the left ventricle LV) such that a distal end 138 of the delivery catheter 136 is adjacent the endocardium 102. In some implementations, the delivery catheter 136 is concentric with the piercing device 130. In other implementations, however, the delivery catheter 136 may not be concentric with the piercing device 130. In yet other implementations, the delivery catheter 136 can be omitted. For example, the device 120 can be delivered directly through or over the piercing device 130, rather than through a separate catheter (see FIGS. 17-19).

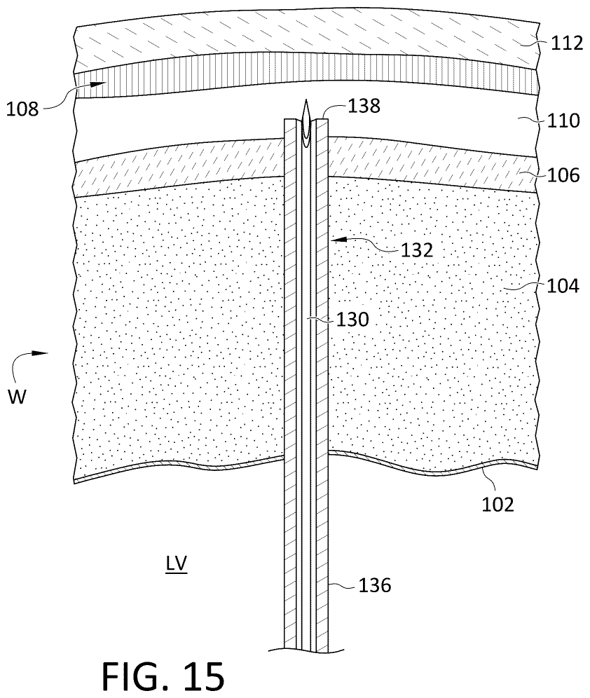

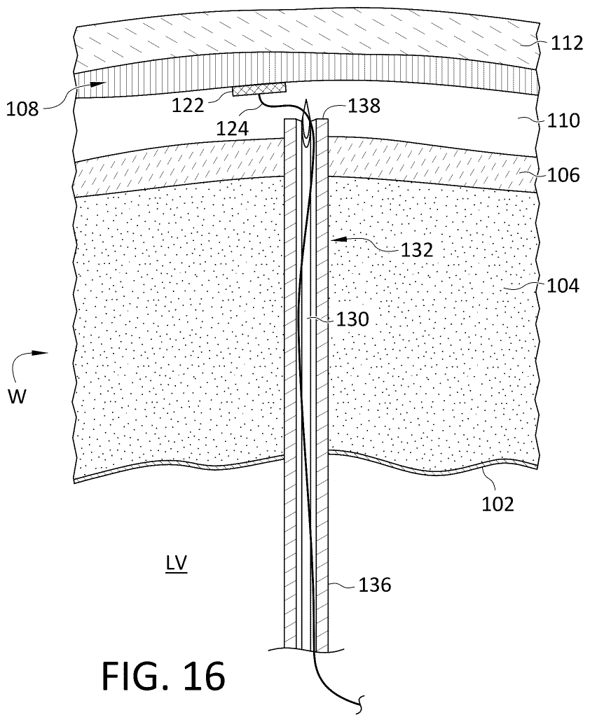

In FIG. 15, the delivery catheter 136 is extended through the passage 132 such that the distal end 138 of the delivery catheter 136 is positioned within the pericardial cavity 110. Referring to FIG. 16, with the distal end 138 of the delivery catheter 136 is positioned within the pericardial cavity 110, the device 120 (see FIG. 20) for remodeling the shape of a heart wall W can be delivered through the delivery catheter 136. In particular, the anchor 122 (see FIGS. 16 and 20) can be extended or pushed out of the distal end of the 138 of the delivery catheter 136 and into the pericardial cavity 110 while the attached line 124 (see FIGS. 16 and 20) extends through the delivery catheter 136 in the passage 132.

As is mentioned above, the anchor 122 can take a wide variety of different forms and can be delivered in a wide variety of different ways. In the examples illustrated by

FIGS. 17-19, the delivery catheter 136 is omitted. In FIG. 17, the anchor 122 is delivered through the piercing device 130 and the catheter 136 can be omitted. The anchor 122 can be delivered through the piercing device 130 in any of the implementations disclosed herein. In

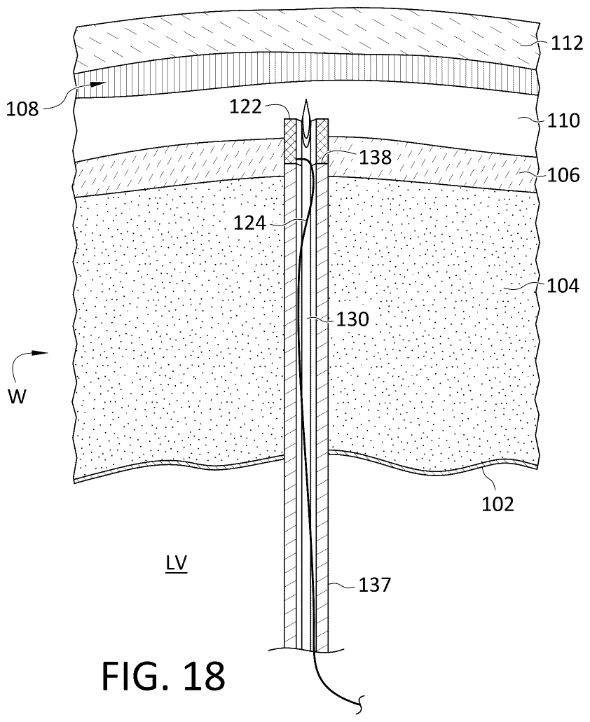

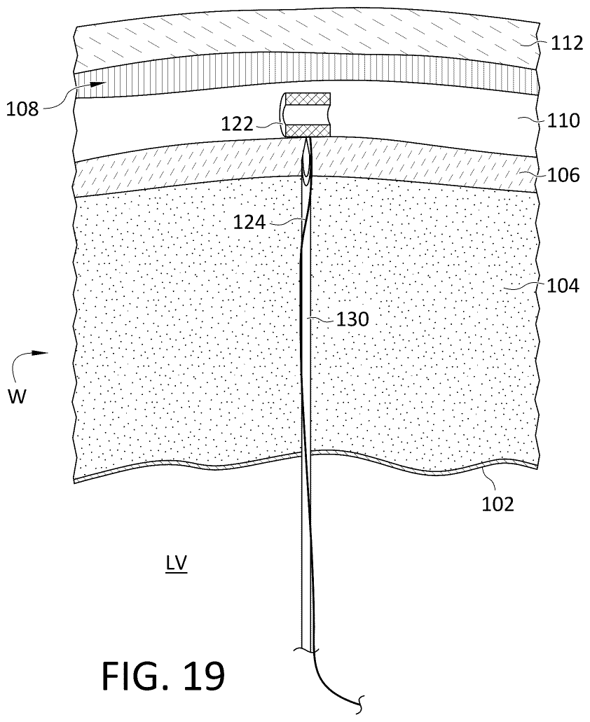

FIG. 18, the anchor 122 is delivered over the piercing device 130. In this example, a pusher 137 pushes the anchor 122 along the outside surface of the piercing device 130 to deploy the anchor 122 as illustrated by FIG. 19. The anchor 122 can be delivered over the piercing device 130 in any of the implementations disclosed herein.

As shown by comparing FIGS. 16 and 20, the delivery catheter 136 and the piercing device 130 can be removed by withdrawing them from the passage 132 and from the heart chamber. The device 120 is left deployed in the heart wall W with the anchor 122 within the pericardial cavity 110 and the line 124 extending through the epicardium 106, the myocardium 104, and the endocardium 102 and into the heart chamber (e.g., the left ventricle LV or the right ventricle RV).

To seat the anchor 122 and to remodel the heart wall W, the line 124 can be placed in tension by pulling the line 124 in an inward direction toward the heart chamber as shown by the arrow A1 in FIG. 20. As will be described in more detail below, two or more devices can be deployed, coupled, and pull against one another to pull the heart wall inward and remodel the shape of the heart wall(s). The anchor 122 will engage and press in on the outward facing surface 126, which in the illustrated implementations is the epicardium 106. The anchor 122 (see FIG. 20) in its deployed state is too large to fit through the passage 132 (see FIG. 14) formed by the piercing device 130 (see FIG. 14). Thus, further tensioning of the line 124 can pull the heart wall W inward toward the heart chamber (e.g., the left ventricle LV).

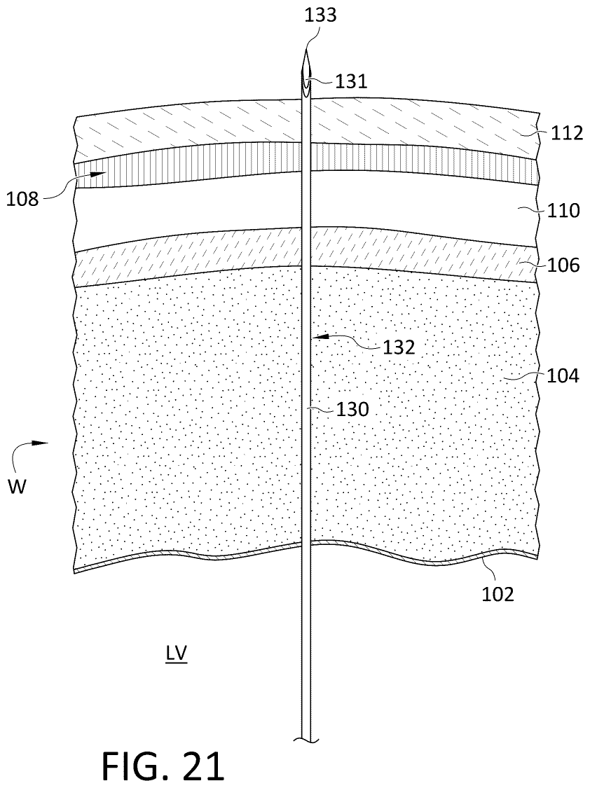

Referring to FIGS. 21-25, an example of a device 120 (FIG. 25) for remodeling the shape of a heart wall W, and an example of a system and method for delivering and deploying the device 120 against an exterior surface of the heart H is illustrated. In some implementations, deployment of the device 120 includes delivering the piercing device 130 into an internal chamber (e.g., left ventricle LV) of the heart H and adjacent the heart wall W, as shown and described in FIG. 8.

In FIG. 21, the piercing device 130 is extended into the heart wall W through the endocardium 102, the myocardium 104, the pericardium 108, and the external parietal pericardium tissue 112 to create a passage 132 through the heart wall W, such that the opening 131 and the distal end 133 of the piercing device 130 are external to the heart H. Thus, the piercing device 130 in FIG. 21 is properly positioned for deploying the anchor 122 in this implementations.

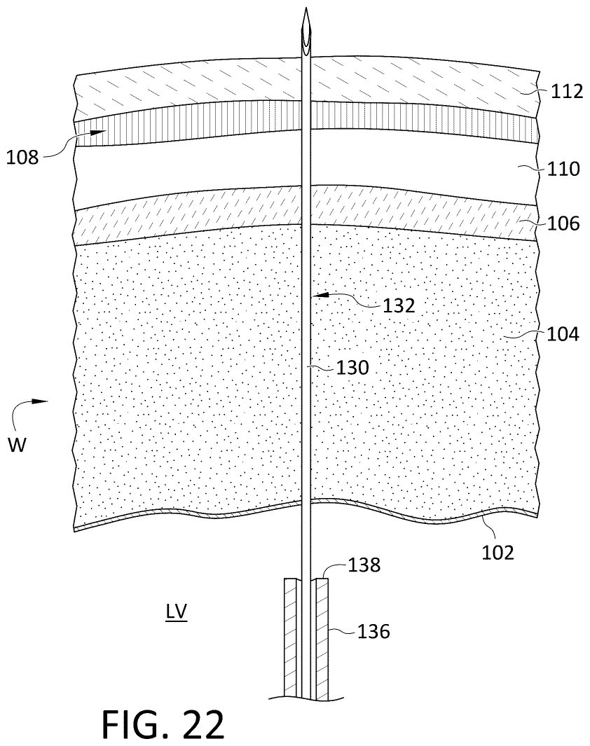

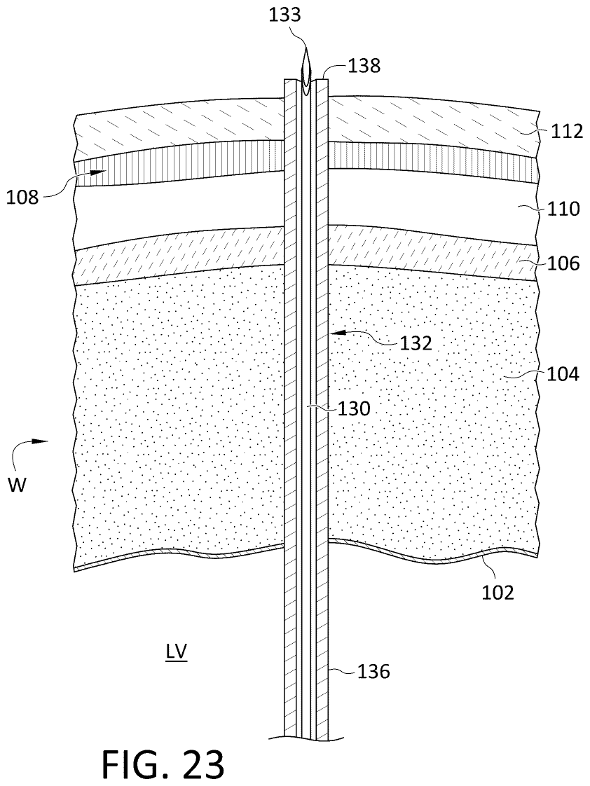

In FIG. 22, the piercing device 130 is extended though the heart wall W and the delivery catheter 136 is positioned within the heart chamber (e.g., the left ventricle LV) such that the distal end 138 of the delivery catheter 136 is adjacent the endocardium 102. In FIG. 23, the delivery catheter 136 is extended through the passage 132 such that the distal end 138 of the delivery catheter 136 is external the heart H, such as adjacent the distal end 133 of the piercing device 130. In some implementations, the delivery catheter 136 is concentric with the piercing device 130. In other implementations, however, the delivery catheter 136 may not be concentric with the piercing device 130. In yet other implementations, the delivery catheter 136 can be omitted. For example, the device 120 can be delivered directly through the piercing device 130, rather than through a separate catheter.

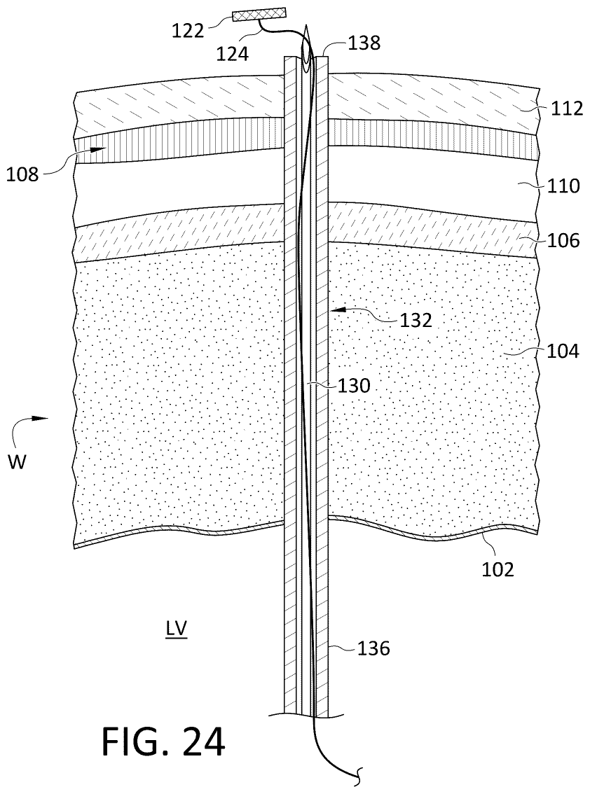

In FIG. 24, the device 120 (FIG. 25) for remodeling the shape of a heart wall W can be delivered through the delivery catheter 136. In particular, the anchor 122 can be extended out of the distal end of the 138 of the delivery catheter 136 external to the heart wall H while the attached line 124 extends through the delivery catheter 136 in the passage 132.

As shown in FIG. 25, the delivery catheter 136 and the piercing device 130 can be removed by withdrawing them from the passage 132 and from the heart chamber. The device 120 is left deployed through heart wall W with the anchor 122 external to the heart wall W and the line 124 extending through the parietal pericardium tissue 112, the pericardium 108, the pericardial space 110, the epicardium 106, the myocardium 104, and the endocardium 102 and into the heart chamber (e.g., the left ventricle LV).

To seat the anchor 122 and to remodel the heart wall W, the line 124 can be placed in tension by pulling the line 124 in an inward direction toward the heart chamber as shown by the arrow A5 in FIG. 25. As will be described in more detail below, two or more devices can be deployed, coupled, and pulled toward one another to pull the heart wall inward and remodel the shape of the heart wall(s). The anchor 122 will engage the outward facing surface 126, which in the illustrated implementation is the exterior parietal pericardium tissue layer 112. In some implementations, the anchor 122 presses a localized area of the pericardium 108 against the epicardium and thus pushes the myocardium 104 inward to remodel the heart wall W. The anchor 122 in its deployed state is too large to fit through the passage 132 formed by the piercing device 130. Thus, further tensioning of the line 124 can pull the heart wall W inward toward the heart chamber (e.g., the left ventricle LV).

In some implementations, the device 120 is configured to prevent blood leakage through the passage 132 into the pericardial space 110. The blood leakage can be blocked in a wide variety of different ways. For example, the anchor 122 can locally pull the pericardium into contact with the epicardium to block the hole through the epicardium, the anchor 122 can cover the hole through the epicardium, a seal separate from the anchor 122 can be placed over the hole in the epicardium, a seal can be placed over the hole in the endocardium, a portion of the anchor 122 can plug the passage 132, the line 124 can be configured to plug the passage, and/or a component disposed over the line can plug the passage. In some implementations, the piercing device 130 and/or the delivery catheter 136 are configured such that the passage 132 and/or the hole in the pericardium closes immediately or substantially immediately upon withdrawal of the piercing device 130 and/or the delivery catheter 136.

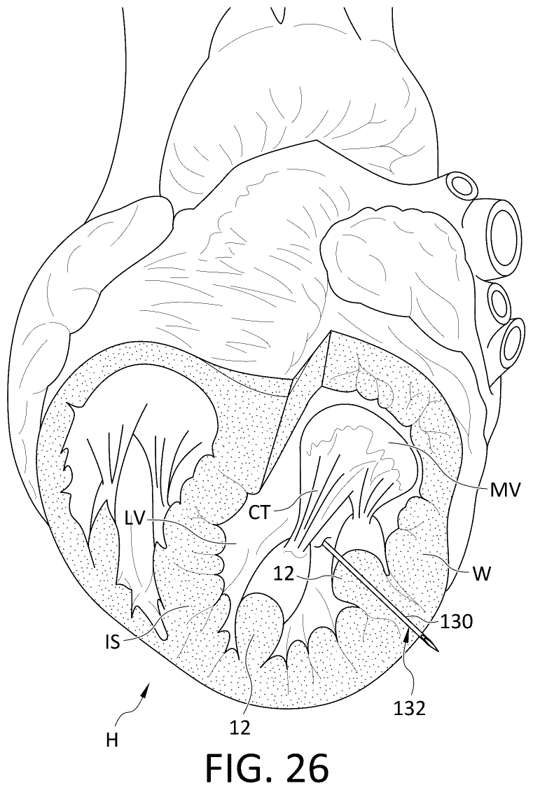

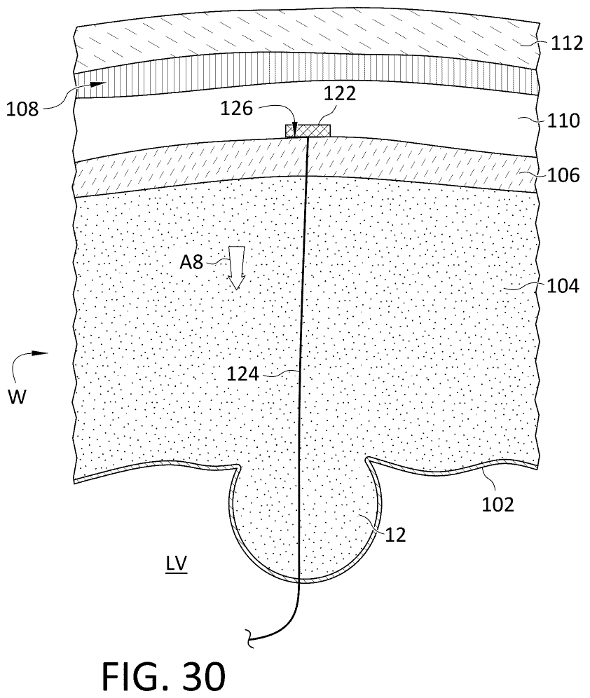

Referring to FIGS. 26-31, another example of deployment of the device 120 for remodeling the shape of a heart wall W and system and method for delivering the device 120. The example illustrated by FIGS. 26-31 can deploy the anchor 122 within the pericardial cavity 110 as illustrated by FIG. 30 or outside the pericardium (see, for example FIG. 25—location can also be selected to pass the line 124 through the papillary muscle). Referring to FIG. 26, in some implementations, deployment of the device 120 includes delivering the piercing device 130 into an internal chamber (e.g., left ventricle LV or right ventricle RV) of the heart H. The piercing device 130 is then extended through one of the papillary muscles 12 and through the heart wall W to create a passage 132.

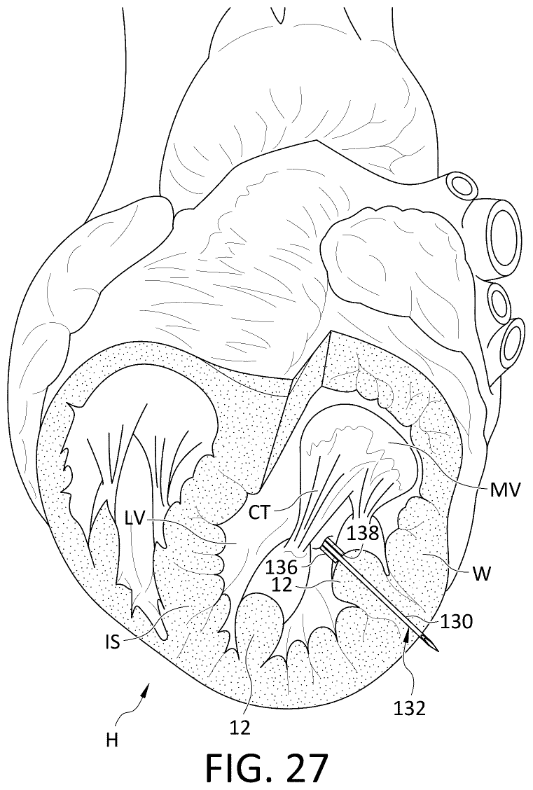

In FIG. 27, the delivery catheter 136 is disposed around the piercing device 130 and positioned within the heart chamber (e.g., the left ventricle LV). The distal end 138 of the delivery catheter 136 is positioned adjacent the papillary muscle 12. In FIGS. 26 and 27, the pericardium 108 is not illustrated. As is noted above, in some implementations the anchor 122 can be deployed into the pericardial space 110 or onto the outside of the pericardium.

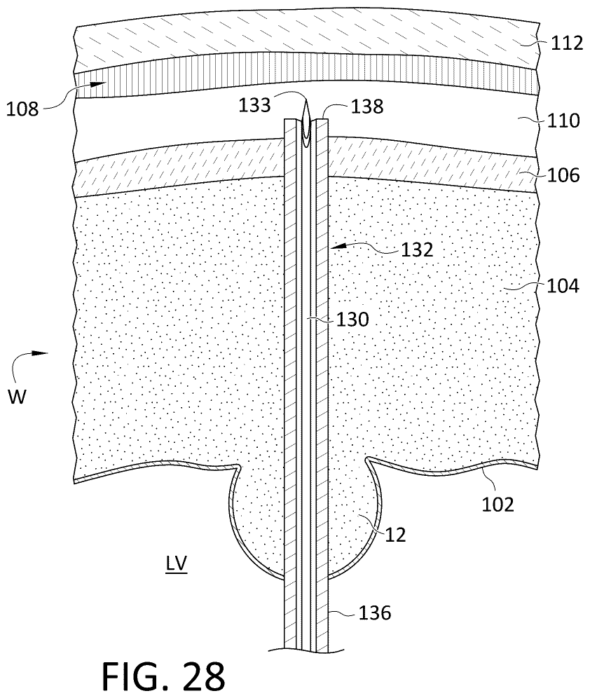

In FIG. 28, the piercing device 130 is illustrated in the proper position for deploying the anchor 122 in the pericardial cavity 110. The delivery catheter 136 is extended through the passage 132 such that the distal end 138 of the delivery catheter 136 is within the pericardial cavity 110 adjacent the distal end 133 of the piercing device 130.

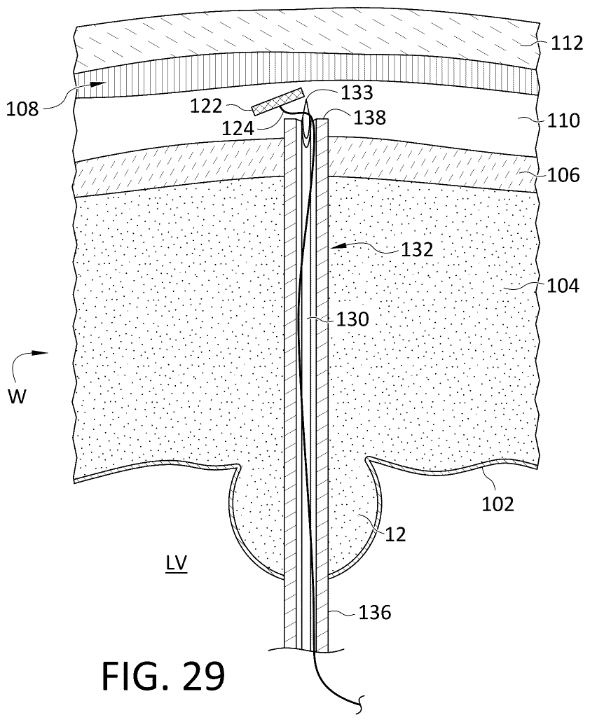

In FIG. 29, the device 120 for remodeling the shape of a heart wall W can be delivered through the delivery catheter 136. In particular, the anchor 122 can be extended out of the distal end of the 138 of the delivery catheter 136 into the pericardial cavity 110 while the attached line 124 extends through the delivery catheter 136 in the passage 132 through the heart wall and the papillary muscle 12.

As shown in FIG. 30, the delivery catheter 136 and the piercing device 130 can be removed by withdrawing them from the passage 132 and from the heart chamber. The device 120 is left deployed through heart wall W with the anchor 122 within the pericardial cavity 110 and the line 124 extending through the epicardium 106, the myocardium 104, and the endocardium 102 and into the heart chamber (e.g., the left ventricle LV).

To seat the anchor 122 and to remodel the heart wall W, the line 124 can be placed in tension by pulling the line 124 in an inward direction toward the heart chamber as shown by the arrow A8 in FIG. 30. As will be described in more detail below, two or more devices can be deployed, coupled, and pulled toward one another to pull the heart wall inward and remodel the shape of the heart wall(s). The anchor 122 will engage and press in on the outward facing surface 126, which in the illustrated implementation is the epicardium 106. The anchor 122 in its deployed state is too large to fit through the passage 132 formed by the piercing device 130. Thus, further tensioning of the line 124 can pull the heart wall W inward toward the heart chamber (e.g., the left ventricle LV).

In some implementations, the device 120 is configured to prevent blood leakage through the passage 132 into the pericardial space 110. The blood leakage can be blocked in a wide variety of different ways, as described herein.

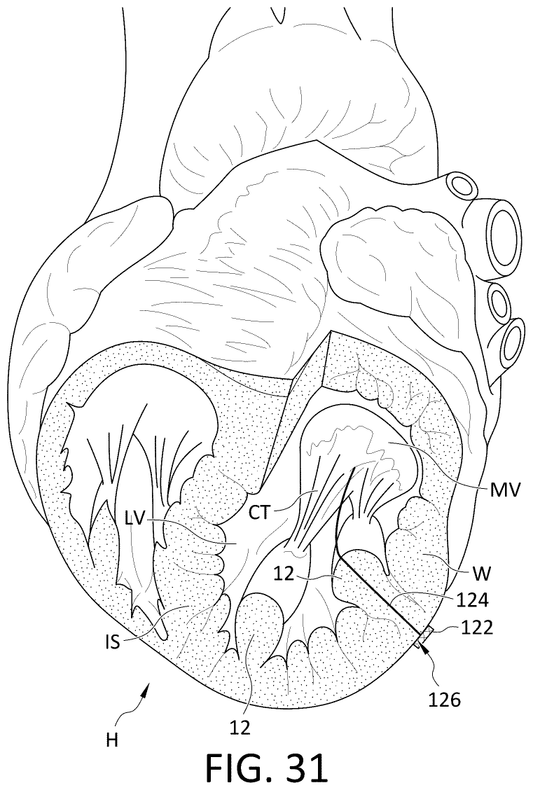

In FIG. 31, the device 120 is illustrated in an installed position with the anchor 122 engaging the outward facing surface 126 and the line extending through the heart wall W and one of the papillary muscles 12 and into the left ventricle LV and through the mitral valve MV. The device illustrated by FIG. 31 can be installed in any manner described herein. In the example illustrated by FIGS. 26-31, the device 120 is installed without an anchoring catheter. However, in other implementations, the device 120 can be installed using an anchoring catheter in any of the manners described herein.

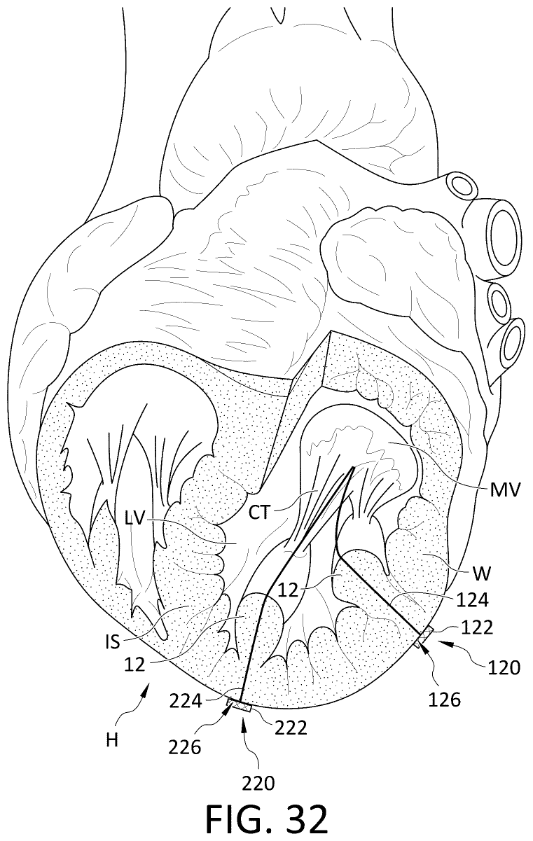

In FIG. 32, along with the installed first device 120, a second device 220 is illustrated in an installed position. The second device 220 includes a second anchor 222 engaging a second outward facing surface 126 and a second line extending through the heart wall W and through another of the papillary muscles 12 and into the left ventricle LV and through the mitral valve MV. The second device 120 can be installed in any of the manners described herein. In on some implementations, the second device 220 is installed in the same manner as the first device 120.

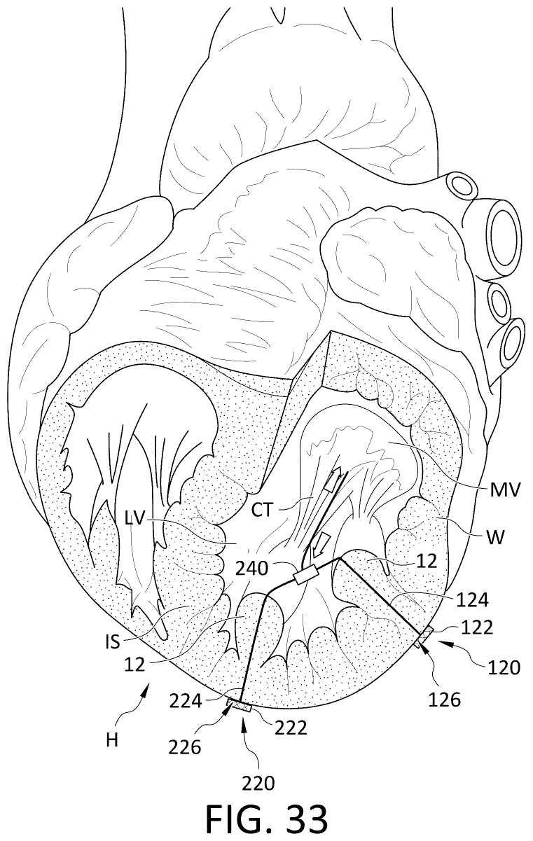

Referring to FIG. 33, in some implementations the first and second lines are routed through a connector 240. In the example illustrated by FIG. 33, the papillary muscles 12 are pulled together or approximated by pushing or holding the position of the connector 240 and pulling on the lines 124, 224. The distance the connector 240 is pushed and the distances the lines 124, 224 are pulled controls how far the papillary muscles 12 are pulled toward one another, which in turn determines the remodeling of the heart walls.

Still referring to FIG. 33, in some implementations, the papillary muscles 12 can be pulled toward one another in a manner that improves coaption between the leaflets of the mitral valve MV. That is, the chordae tendinea are attached to the mitral valve MV leaflets and the papillary muscles. Approximating the papillary muscles toward one another causes the chordae tendinea CT to pull the mitral valve leaflets toward one another and enhance coaption of the mitral valve leaflets. The enhanced or corrected leaflet coaption can reduce or eliminate mitral valve regurgitation.

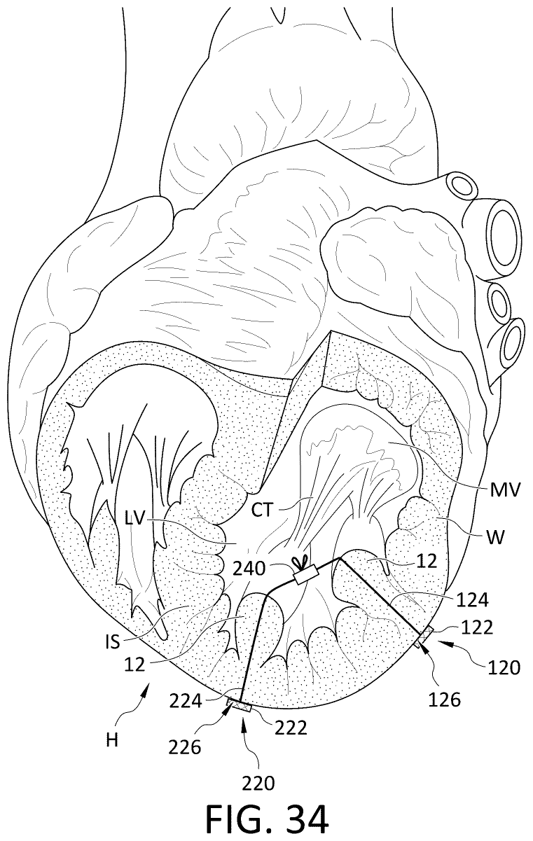

Referring to FIG. 34, after the papillary muscles 12 and the heart walls W are pulled to the desired remodeled position by the lines 124, 224, the connector 240 secures the positions of the lines in the connector. Once secured, the lines can be trimmed as shown. As such, both the device 120 and the second device 220 are shown in installed positions with the anchors 122, 222 engaging the outward facing surfaces 126, 226 and the lines 124, 224 extending through the heart wall W, through papillary muscles 12 and into the left ventricle LV. The lines 124, 224 remain in tension to pull inward to pull the heart wall W inward to remodel the shape of the heart wall W.

The lines 124, 224 can be connected together in a variety of ways. For example, the lines can be tied together or held together by a line locking device 240. The line locking device 240 can be any suitable device that can hold the lines 124, 224 together in tension such that the heart wall W is held in the remodeled position. A variety of different line locking devices 240 are shown and described below.

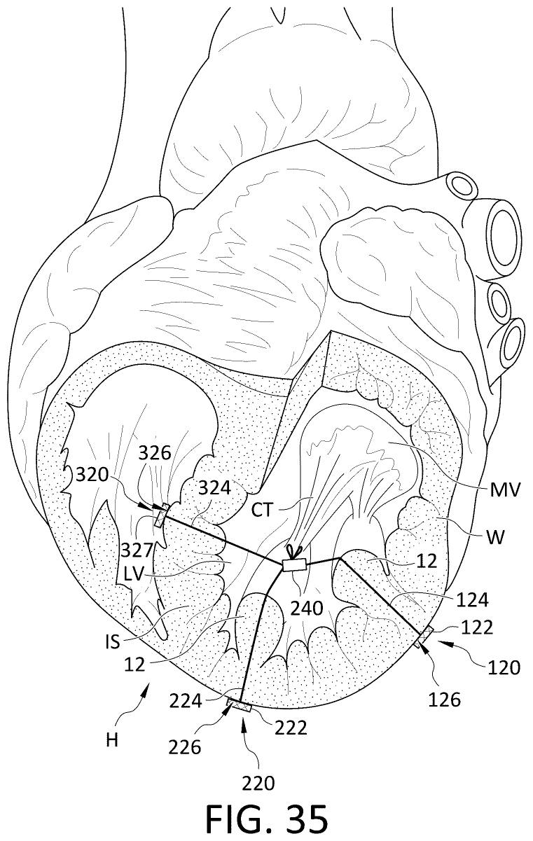

Any number of devices 120 and any number of line locking devices 240 can be used to tailor the heart wall remodeling to each individual patient. In some implementations, two or more lines 124 are locked together in each of the locking devices. FIG. 35 illustrates one example where three lines are connected together by one locking device. In some implementations, more than one locking device is used, with at least two lines being connected together by each locking device. In the example illustrated by FIG. 35, along with the installed first device 120 and a second device 220, a third device 320 is illustrated in an installed position with a third anchor 327 engaging a third outward facing surface 326 and a third line 324 extending through a heart wall and into the left ventricle LV. In the illustrated example of FIG. 35, the heart wall associated with the third device 320 is the interventricular septum IS. Thus, the interventricular septum IS defines the third outward facing surface 326 and the third line 324 extends through the interventricular septum IS.

As with the devices of FIG. 34, the lines 124, 224, 324 can be pulled inward to pull the heart wall W inward to remodel the shape of the heart wall W. To hold the heart wall W in the remodeled position, the lines 124, 224, 324, while in tension, can be connected within the left ventricle LV, such as for example, by the line locking device 240 or other suitable means for connecting the lines 124, 224, 324.

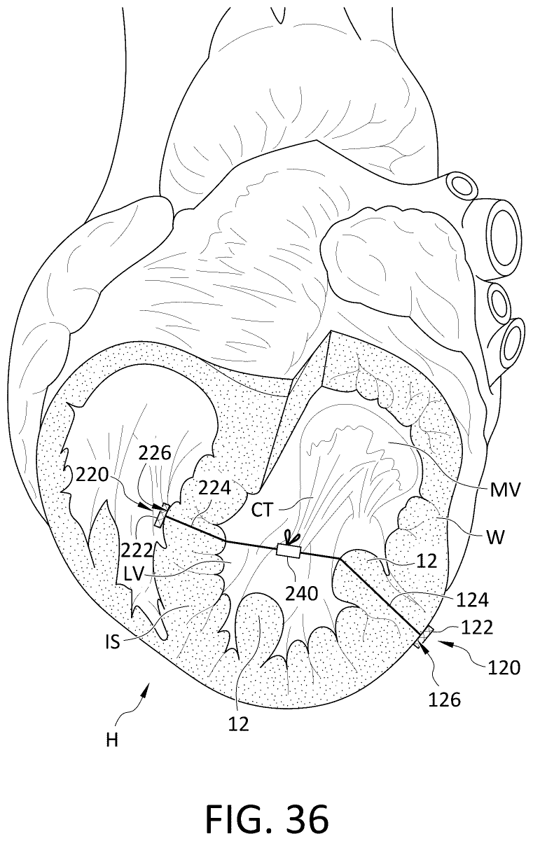

In FIG. 36, both the device 120 and the second device 220 are shown in installed positions with the anchors 122, 222 engaging the outward facing surfaces 126, 226. The line 124 extends through the heart wall W, through a papillary muscle 12 and into the left ventricle LV. The second device 220 is installed such that the second anchor 222 engages the outward facing surface 226 on the interventricular septum IS and the second line 224 extends through the interventricular septum IS and into the left ventricle LV.

As with the devices of FIG. 34, the lines 124, 224, can be pulled inward to pull the heart wall W inward and to pull the interventricular septum IS inward to remodel the shape of the heart wall W and the interventricular septum IS. To hold the heart wall W and the interventricular septum IS in the remodeled positions, the lines 124, 224, while in tension, can be connected within the left ventricle LV, such as for example, by the line locking device 240 or other suitable means for connecting the lines 124, 224.

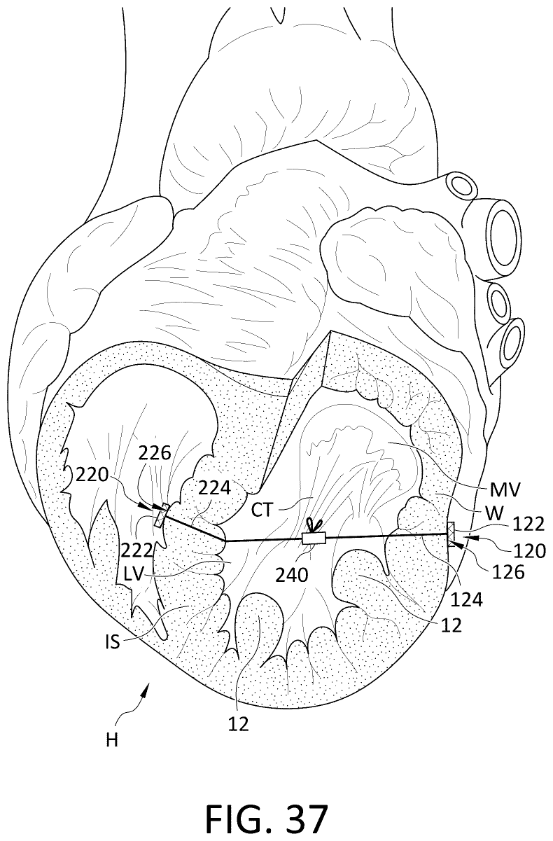

In FIG. 37, both the device 120 and the second device 220 are shown in installed positions with the anchors 122, 222 engaging the outward facing surfaces 126, 226. The line 124 extends through the heart wall W and into the left ventricle LV, but not through a papillary muscle 12. The second device 220 is installed such that the second anchor 222 engages the outward facing surface 226 on the interventricular septum IS and the second line 224 extends through the interventricular septum IS and into the left ventricle LV.

As with the devices of FIG. 34, the lines 124, 224, can be pulled inward to pull the heart wall W inward to remodel the shape of the heart wall W and the interventricular septum IS. To hold the heart wall W and the interventricular septum IS in the remodeled position, the lines 124, 224, while in tension, can be connected within the left ventricle LV, such as for example, by the line locking device 240 or other suitable means for connecting the lines 124, 224.

The devices 120 can be used in a wide variety of different ways to remodel the heart and/or approximate the papillary muscles. A single locking device 240 and two or more devices 120 can be deployed or more than one line locking device 240 with two or more devices 120 per locking device 240 can be deployed to remodel the heart of a single patient. For example, any of the configurations illustrated by FIGS. 34-37 can be used in combination on the heart of a single patient. For example, one pair of devices 120, 220 can be used to approximate the papillary muscles 12, while one or more additional pairs (or three, or four, etc.) of devices can be used to remodel the shape of the right ventricle, in the same patient. Or, in some implementations, one pair of devices 120, 220 pulls one papillary muscle and a portion of the heart wall or interventricular septum IS relatively toward one another and another on pair of devices 120, 220 pulls another papillary muscle and a portion of the heart wall or interventricular septum IS relatively toward one another. In another implementation, the lines are not deployed through the papillary muscles and one pair of devices 120, 220 pulls two portions of the heart wall or interventricular septum IS relatively toward one another and another pair of devices 120, 220 pulls two other portions of the heart wall or interventricular septum IS relatively toward one another.

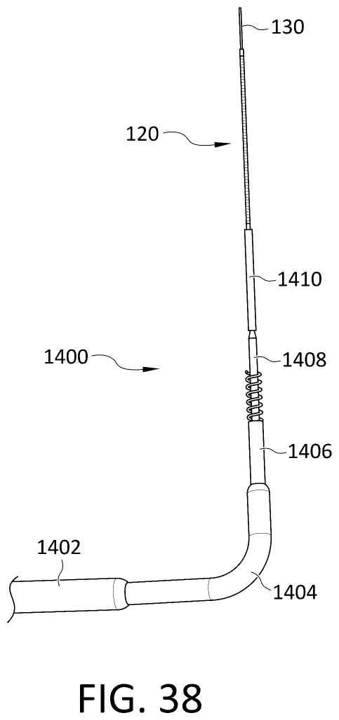

Referring to FIGS. 38-55, an example of a device 120 for remodeling the shape of a heart wall and a system 1400 and method for delivering the device 120 against an exterior surface of the heart H is illustrated. The system 1400 and the device 120 can be configured in a variety of ways.

In the example illustrated in FIG. 38, the system 1400 includes a guide sheath 1402, a steerable catheter 1404, a delivery catheter 1406, a pusher 1408, a hemostatic plug 1410, a piercing device 130, and the device 120.

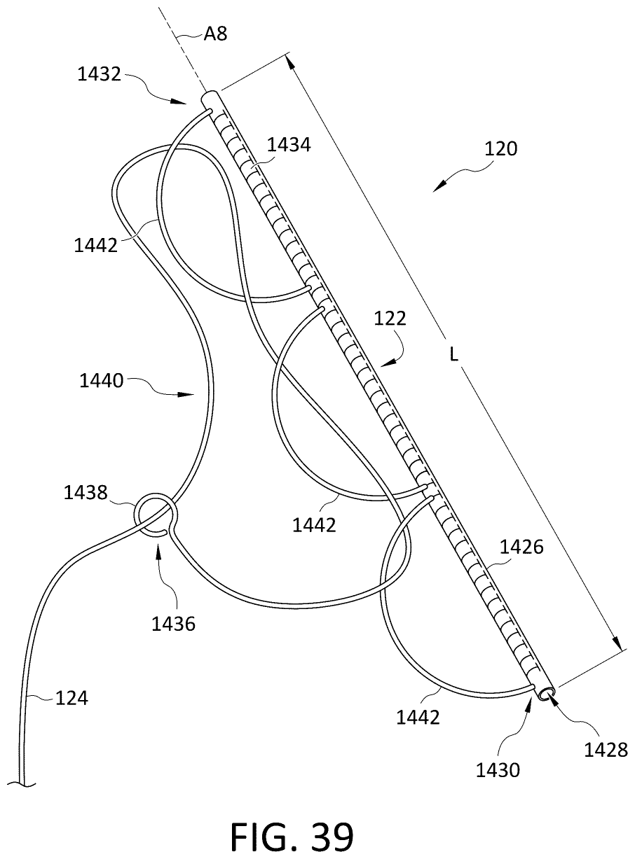

Referring to FIG. 39, an example of the device 120 for remodeling the shape of a heart wall is illustrated. The device 120 includes an anchor 122 and a line 124. The anchor 122 can be configured in a variety of ways. Any configuration that can be positioned to engage an outward facing surface of a heart wall W to support pulling a portion of the heart wall W inward (i.e., toward an internal chamber of the heart) can be used. In some implementations, the anchor 122 is reconfigurable, such that the anchor can be delivered through a catheter or sheath in a delivered state (e.g., elongated as shown in FIG. 39) that fits within a lumen of delivery catheter 1406, and can be reshaped to a deployed state once it has been delivered to the appropriate location.

In some implementations, the anchor 122 has a generally cylindrical elongated body 1426 forming a tube having a central passage 1428. In other implementations, however, the body 1426 can be shaped other than cylindrical. For example, the elongated body 1426 can have an oval, rectangular, or other shaped cross section. The body 1426 has a length L and includes a first end portion 1430 and a second end portion 1432 opposite the first end portion.

In some implementations, the body 1426 includes one or more features to facilitate bending of the body 1426. The features can be configured in a variety of ways. In some implementations, the features include a series of traverse cuts 1434 along the body 1426. In some implementations, the series of cuts 1434 are a plurality of cuts where each of the cuts is generally perpendicular to a longitudinal axis A8 of the body 1426. In some implementations, the cuts in the series of cuts 1434 are evenly spaced along the body 1426 and extend from the first end portion 1430 to the second end portion 1432. For example, the series of cuts 1434 can extend over at least 80% of the length L of the body M. In other implementations, the series of cuts 1434 can unevenly spaced and/or can extend less than 80% of the length L of the body 1426.

Further, each of the cuts of the series of cuts 1434 extends partially into the body 1426. In some implementations, each of the cuts extend between 25%-75% through the body.

The anchor 122 can be made from any suitable material that can be reshaped from an elongated state to a curved, deployed state. In some implementations, the anchor 122 includes a shape-memory alloy—such as Nitinol—to provide shape-setting capability.

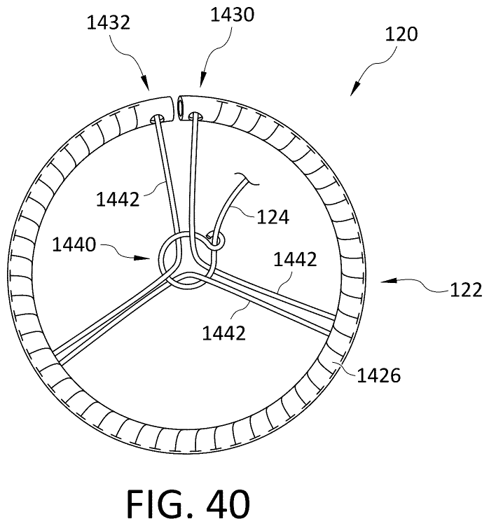

The line 124 is connected to the anchor 122 such that pulling the line 124 can pull the anchor 122. In some implementations, pulling the line 124 can also reshape the anchor from an elongated state (FIG. 39) to a curved, deployed state (FIG. 40). In some implementations, the line 124 has a terminal end 1436 formed in a closed shape 1438, such as a circle. The line 124 is arranged such that the line 124 passes through the closed-shaped terminal end 1436 to form a loop 1440 in the line 124 that can be withdrawn by pulling the line 124.

The line 124 can be connected to the anchor 122 in a variety of ways. In some implementations, the anchor includes a plurality of loops 1442 and the line 124 passes through each of the loops 1442 to connect the line 124 to the anchor 122. The number, size, location, and configuration of the loops 1442 can vary in different implementations. In some implementations, the anchor includes three equal sized and evenly spaced loops 1442 along the length L of the anchor 122.

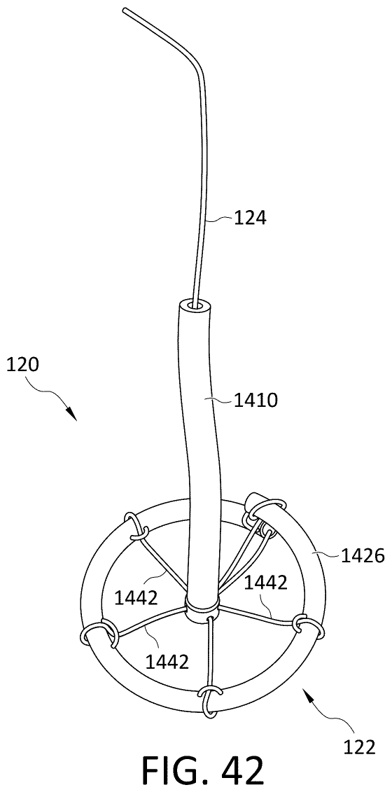

In some implementations, each of the loops 1442 is formed by a line fixedly attached, at its ends, to the body 1426 of the anchor 122. The line forming the loops 1442 can be fixedly attached in any suitable manner. In some implementations, the body 1426 can include openings that accommodate insertion of the ends of the line forming the loops 1442 into the interior of body 1426 to attach the ends to the body 1426 (see, for example, FIG. 40). In other implementations, the lines that form the loops 1442 can be tied to the exterior of the body 1426 of the anchor 122 (see, for example, FIG. 42) or otherwise attached to the exterior, such as for example, by an adhesive or other fastening device.

In other implementations, however, the loops 1442 can be formed from a material other than a line. The loop 1440 in the line 124 passes through each of the loops 1442 to connect the line 124 to the anchor 122.

Referring to FIG. 40, the anchor 122 is illustrated in a deployed state. The deployed state of the anchor 122 can be a variety of shapes. Any shape that can be used to pull a portion of a heart wall inward to remodel the shape of the heart wall can be used, such as for example, a curved shape. In some implementations, the anchor 122 is ring-shaped in the deployed state such that the first end portion 1430 is adjacent the second end portion 1432 and the body 1426 is curved in a circle. In some implementations, the first end portion 1430 and the second end portion 1432 can abut. In other implementations, the first end portion 1430 and the second end portion 1432 may not abut. For example, the first end portion 1430 and the second end portion 1432 can be spaced apart from one another or can overlap,

The anchor 122 can be reshaped from the elongated state to the deployed state in a variety of ways. For example, the anchor 122 can include a shape-memory alloy and be shape set to the shape of the deployed state. Alternatively, or in conjunction with the anchor being shape set, the line 124 can be used to reshape the anchor 122. In particular, since the line loop 1440 passes through each of the loops 1442, by pulling on the line 124, the line loop 1440 is withdrawn which pulls the first end portion 1430 and second end portion 1432 together while bending the body 1426 into an annulus.

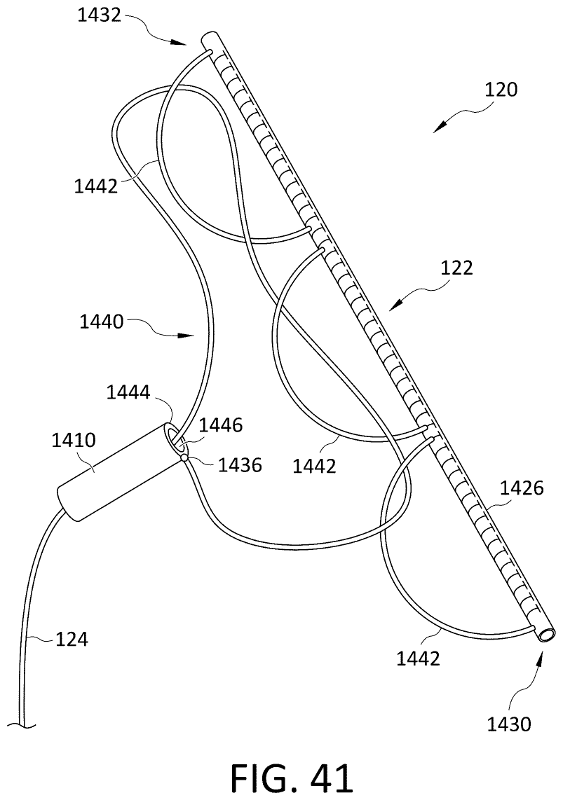

In FIG. 41, the device 120 includes the hemostatic plug 1410. The hemostatic plug 1410 can be configured in a variety of ways. Any device that can stop bleeding from occurring from a passage formed by the piercing device 130 in a heart wall can be used. In some implementations, the hemostatic plug 1410 is formed as a cylindrical tube having a distal end 1444 and a central passage 1446 through which the line 124 extends. In other implementations, however, the hemostatic plug 1410 can be configured other than cylindrical, as will be described in greater detail herein.

In some implementations, the terminal end 1436 of the line 124 is attached to the distal end 1444 of the hemostatic plug 1410. The line 124 forms the loop 1440 and is connected to the anchor 122 by passing through each of the loops 1442.

Referring to FIG. 42, the anchor 122 is illustrated in a deployed state. In some implementations, the anchor 122 is ring-shaped in the deployed state such that the first end portion 1430 is adjacent the second end portion 1432, the body 1426 is curved in a circle, and the distal end 1444 of the hemostatic plug 1410 is generally adjacent the center of the circle.

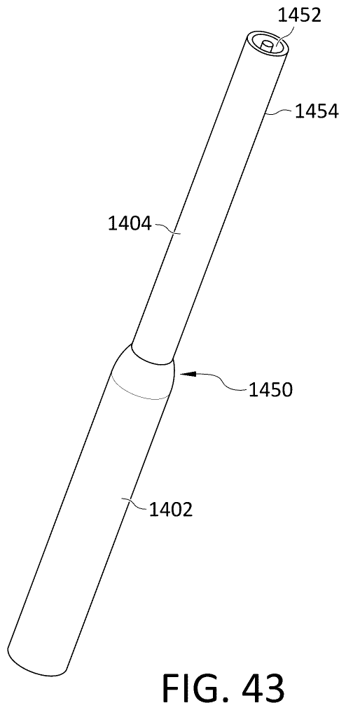

In FIG. 43, an example of a portion of the guide sheath 1402 and a portion of the steerable catheter 1404 is illustrated. Both the guide sheath 1402 and the steerable catheter 1404 can be configured in a variety of ways. Any suitable known guide sheath 1402 and steerable catheter 1404 can be used. In some implementations, the guide sheath 1402 and the steerable catheter 1404 are concentric where the guide sheath 1402 includes an inner lumen (not shown) and the steerable catheter extends through the inner lumen and out of a distal end 1450 of the guide sheath 1402. Likewise, the steerable catheter 1404 includes an inner lumen 1452 that is open at a distal end 1454 of the steerable catheter 1404.

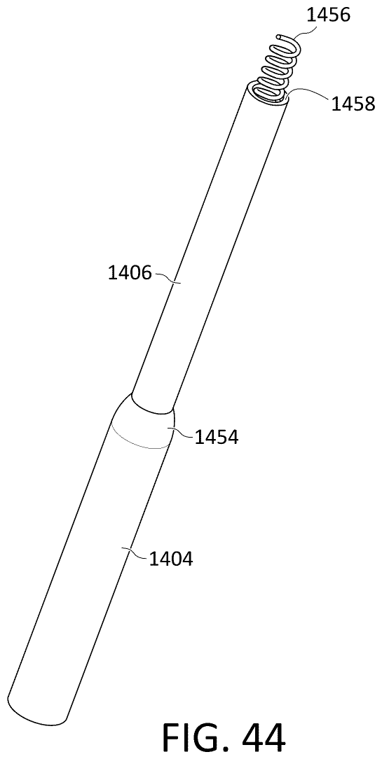

In FIG. 44, a portion of the steerable catheter 1404 and a portion of the delivery catheter 1406 are illustrated. The delivery catheter 1406 includes an anchoring device 1456 attached to a distal end 1458 of the delivery catheter 1406. The anchoring device 1456 can be any suitable device capable of attaching to the heart wall W. In some implementations, the anchoring device 1456 is a wire formed in a helical shape configured to be screwed into the heart wall W to secure the delivery catheter 1406 to the heart wall W. The delivery catheter 1406 includes an inner lumen (not shown) open at the distal end 1458.

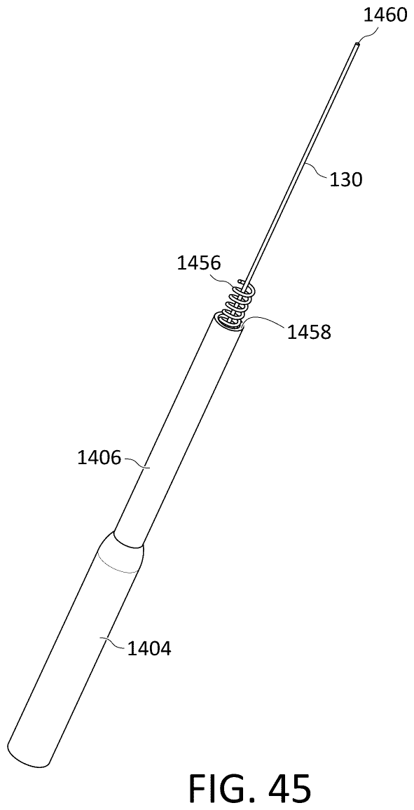

In FIG. 45, a portion of the steerable catheter 1404, a portion of the delivery catheter 1406, and the piercing device 130 are illustrated. The piercing device 130 can be any suitable device for piercing or creating a passage into a human heart wall, such as for example, a needle, wire, or other similar device. In some implementations, the piercing device 130 is a needle or hollow wire having an inner passage (not shown) and an opening (not shown) proximate a distal end 1460 of the piercing device 130 the fluidly connects the inner passage (not shown) to the exterior of the piercing device 130.

In some implementations, the piercing device 130 is delivered through the inner lumen (not shown) of the delivery catheter 1406 and extends out of the distal end 1458 of the delivery catheter 1406.

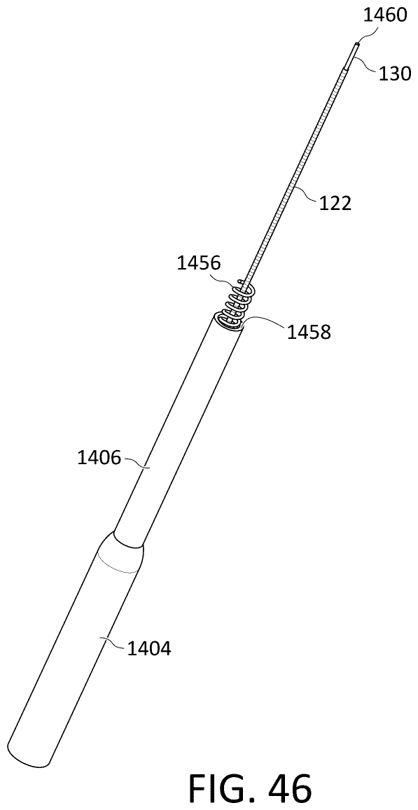

In FIG. 46, a portion of the steerable catheter 1404, a portion of the delivery catheter 1406, the piercing device 130, and the anchor 122 are illustrated. The anchor 122 is shown in the elongated state and it extends from the distal end 1458 of the delivery catheter 1406 over top of the piercing device 130 such that the piercing device 130 is received in the central passage 1428 of the anchor 122 and the anchor 122 and the piercing device 130 are concentric.

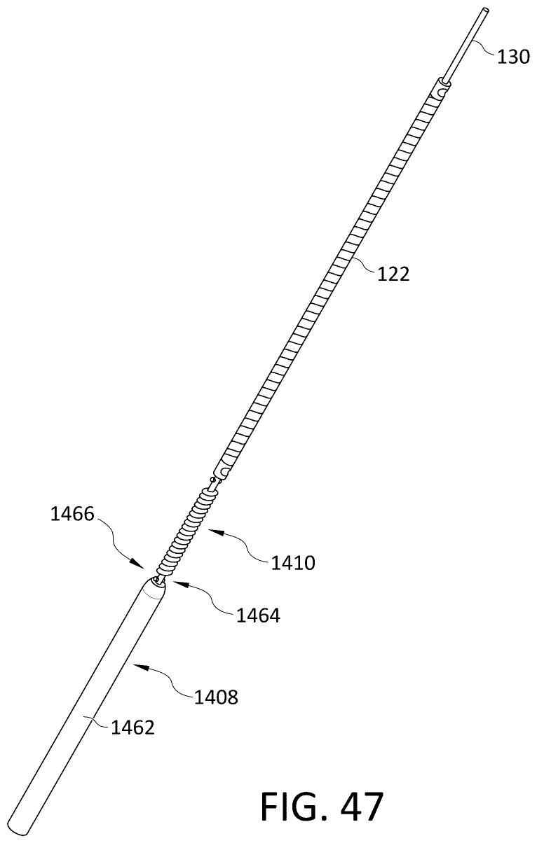

In FIG. 47, examples of the piercing device 130, the anchor 122, the hemostatic plug 1410, and the pusher 1408 are illustrated. The pusher 1408 can be configured in a variety of ways. Any configuration capable of pushing the hemostatic plug 1410 and anchor 122 over the piercing device 130 and beyond the distal end 1460 of the piercing device 130 can be used. In some implementations, the pusher 1408 has an elongated, cylindrical body having a distal end 1464 configured to abut a proximal end 1466 of the hemostatic plug 1410. The body includes an inner passage (not shown) extending through the pusher body. The piercing device 130 can be received in the passage (not shown) such that the pusher 1408 is slidable over the piercing device 130 and concentric with the piercing device 130.

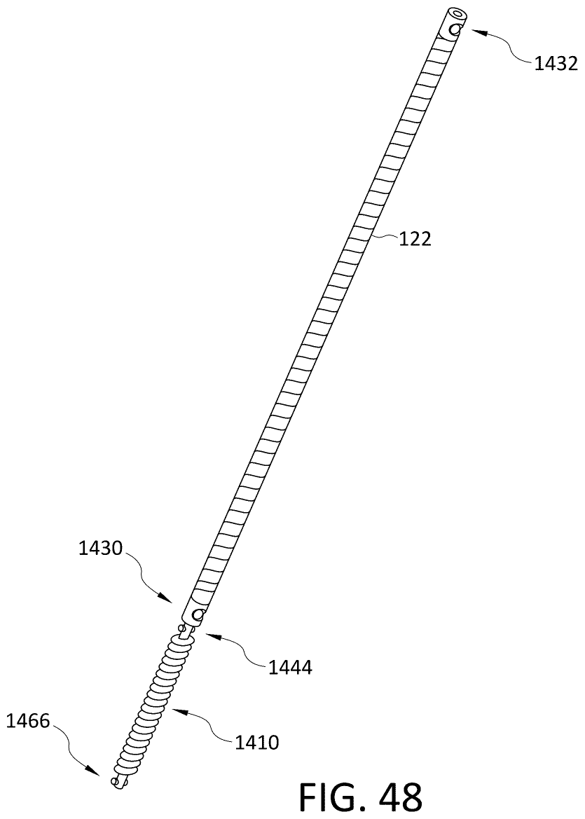

In FIG. 48, the anchor 122 and the hemostatic plug 1410 are illustrated. The anchor 122 is shown in the elongated state. and the hemostatic plug 1410 is illustrated as a cylindrical tube. Both the anchor 122 and the hemostatic plug 1410 are configured to be concentric with and slidable over the piercing device 130. When being delivered over the piercing device 130, the anchor 122 and the hemostatic plug 1410 are longitudinally aligned, with the distal end 1444 of the hemostatic plug 1410 adjacent the first end 1430 of the anchor 122, as shown in FIG. 48.

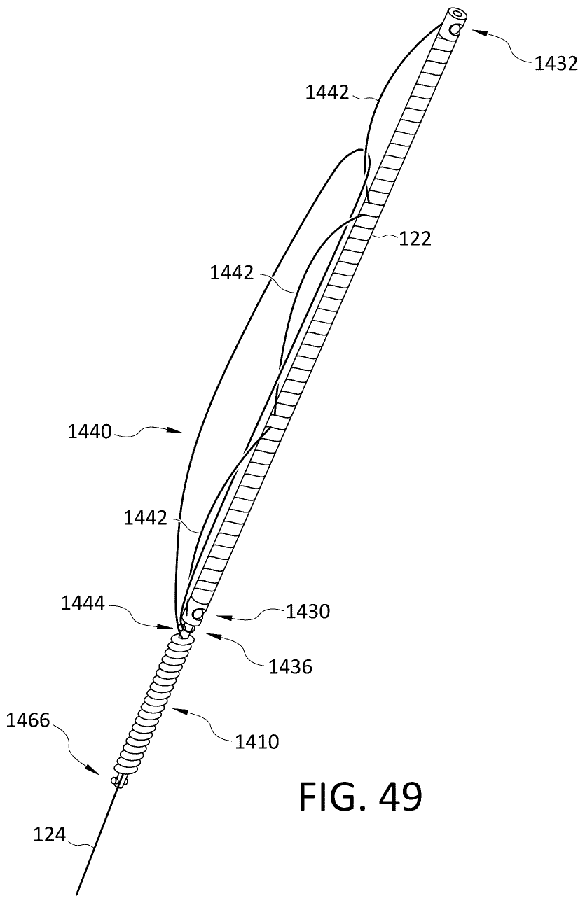

In FIG. 49, the anchor 122 and the hemostatic plug 1410 are illustrated along with the line 124 and the loops 1442 on the anchor 122. The terminal end 1436 of the line 124 is attached to the distal end 1444 of the hemostatic plug 1410. The loop 1440 of the line 124 passes through each of the loops 1442 to connect the line 124 to the anchor 122. The line 124 then extends through the central passage 1446 (FIG. 41) of the hemostatic plug from the distal end 1444 to the proximal end 1466.

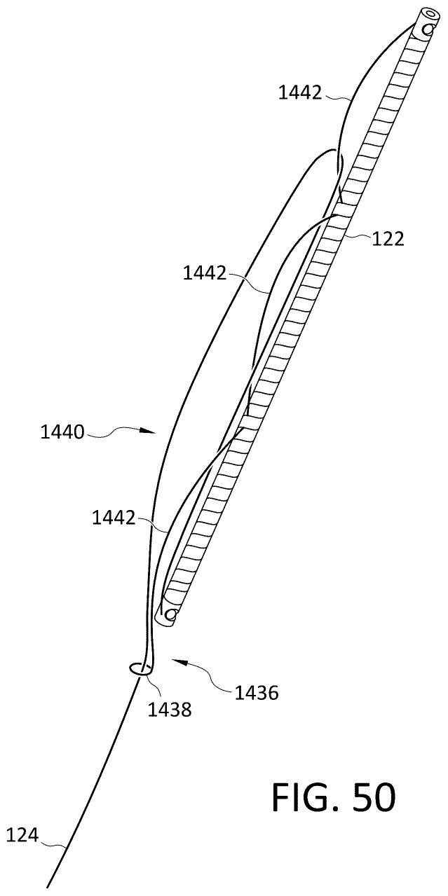

In FIG. 50, the anchor 122 without a hemostatic plug 1410 is illustrated along with the line 124 and the loops 1442 on the anchor 122. The terminal end 1436 of the line 124 is formed in a closed, circular shape 1438 and the line 124 is arranged such that the line 124 passes through the closed-shaped terminal end 1436. The loop 1440 of the line 124 passes through each of the loops 1442 to connect the line 124 to the anchor 122.

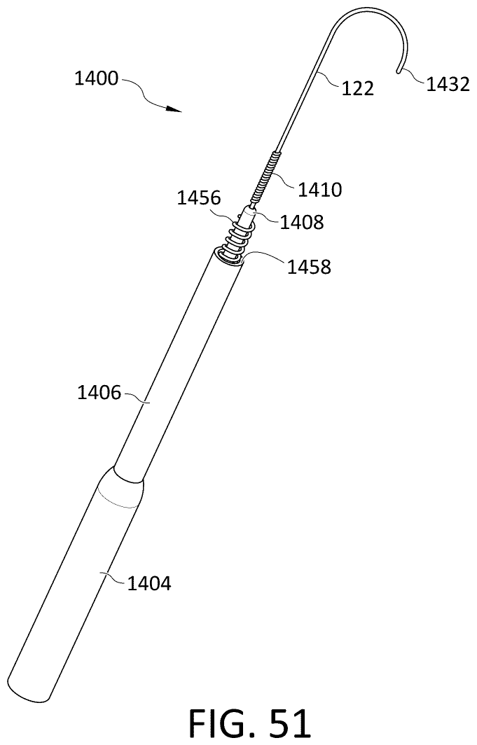

In FIG. 51, the system 1400 is illustrated showing the anchor 122 partially deployed beyond the distal end 1460 of the piercing device 130 (FIG. 45). The delivery catheter 1406 is illustrated partially extending from the steerable catheter 1404 and the pusher 1408 is illustrated partially extending from the delivery catheter 1406 through the anchoring device 1456. As the pusher 1408 extends from the delivery catheter 1406, the distal end 1464 of the pusher 1408 engages the proximal end 1466 of the hemostatic plug 1410 to push both the hemostatic plug 1410 and the anchor 122 over the piercing device 130. As the distal end 1432 of the anchor 122 moves beyond the distal end 1460 of the piercing device 130 (FIG. 45), the anchor 122 can begin to reshape into its curved deployed state. For example, the anchor 122 can include a shape memory alloy that is shape set to the curved deployed state. Thus, the portion of the anchor 122 that is no longer received on the piercing device 130 can revert to the curved deployed state that that shape memory alloy was set to.

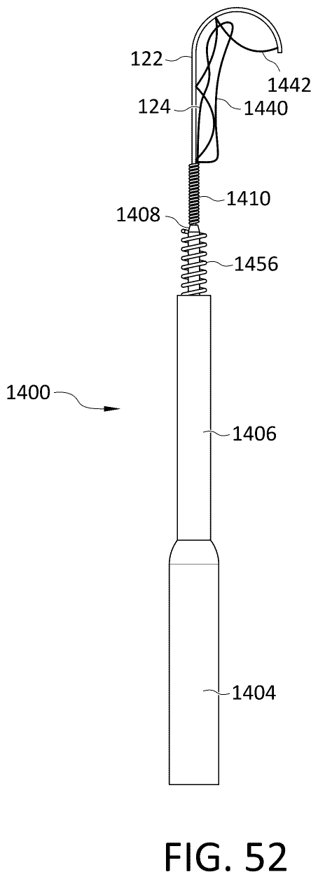

In FIG. 52, the system 1400 is illustrated showing the anchor 122 partially deployed beyond the distal end 1460 of the piercing device 130 (FIG. 45) along with the line 124 connected to the anchor 122. In particular, the line 124 connects to the hemostatic plug 1410, extends through the loops 1442, and then loops around and extend though the central passage 1446 (FIG. 41) of the hemostatic plug 1410.

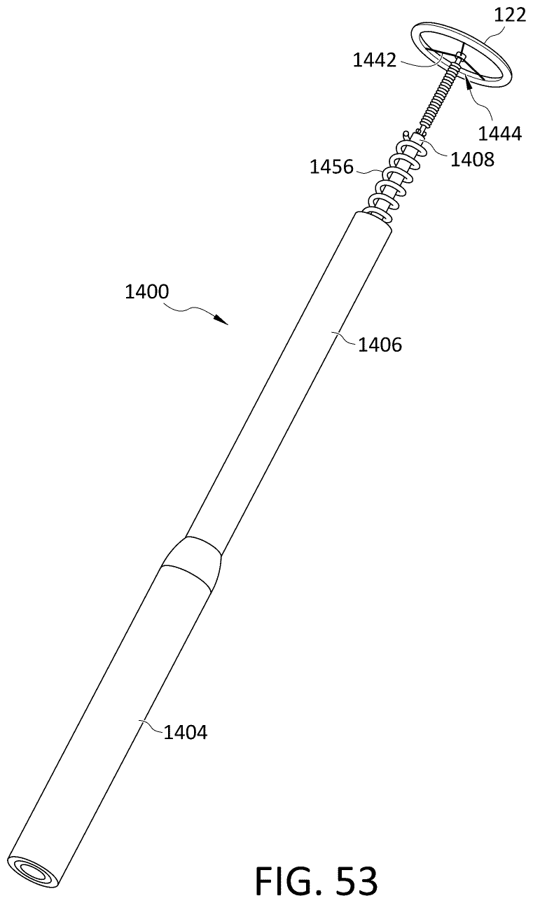

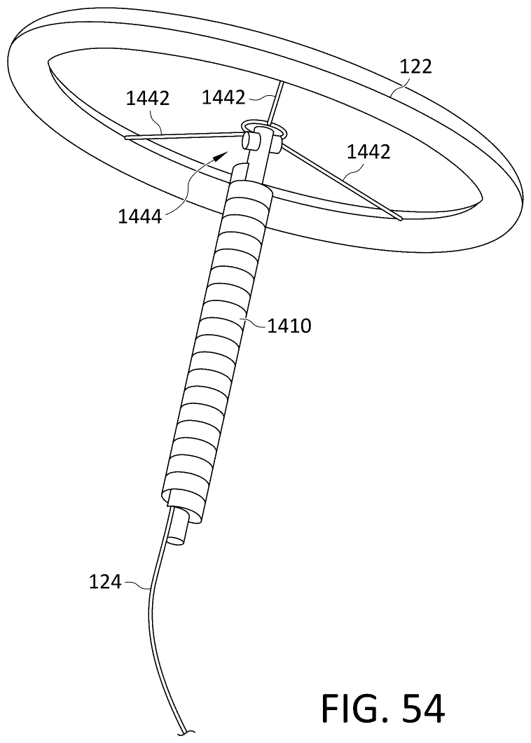

In FIGS. 53-54, the system 1400 is shown with the anchor 122 in the deployed state. In particular, the line 124 (FIG. 54) is withdrawn by pulling the line 124 in a direction away from the anchor 1422 and into the delivery catheter 1406. Since the loop 1440 (FIG. 41) of the line 124 passes through the loops 1442 on the anchor 122, pulling the line 124 closes the loop 1440 and pulls the loops 1442 together to help facilitate, along with any shape setting properties of the anchor, reshaping the anchor 122 from the elongated state to the curved deployed state shown in FIGS. 53-54. At the same time, the distal end 1444 of the hemostatic plug 1410 is positioned adjacent the center of the curved anchor 122.

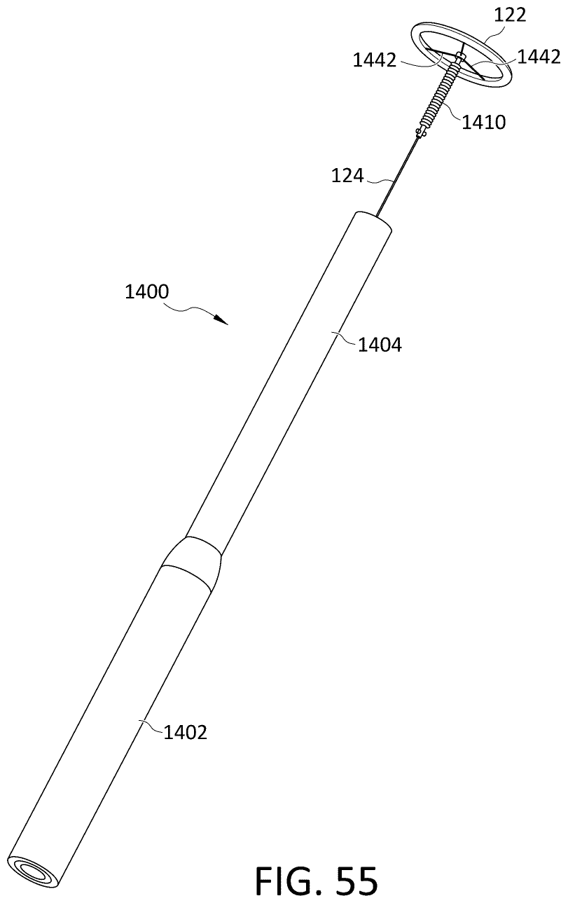

In FIG. 55, the system 1400 is shown with the anchor 122 in the deployed state and the delivery catheter 1406 retracted back into the steerable catheter 1404. The line 124 (see FIG. 54) is withdrawn by pulling the line 124 in a direction away from the anchor 1422 and into the delivery catheter 1406. Since the loop 1440 (FIG. 41) of the line 124 passes through the loops 1442 on the anchor 122, pulling the line 124 closes the loop 1440 and pulls the loops 1442 together to help facilitate, along with any shape setting properties of the anchor, reshaping the anchor 122 from the elongated state to the curved deployed state shown in FIGS. 53-54. At the same time, the distal end 1444 of the hemostatic plug 1410 is pulled up to the center of the curved anchor 122 as the loop 1440 is shortened by pulling the line 124.

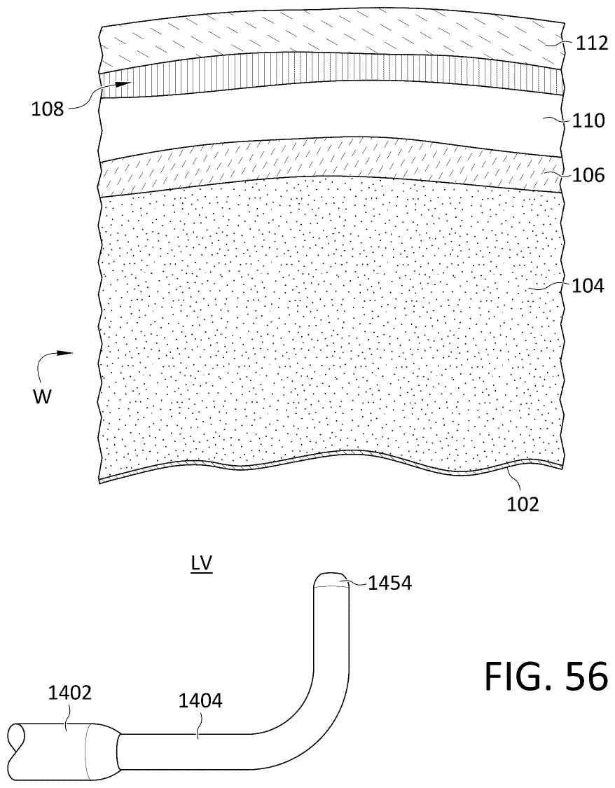

Referring to FIGS. 56-64, the deployment of the device 120 into the pericardial cavity 110 for remodeling the shape of a heart wall W and system and method for delivering the device 120 is illustrated. Referring to FIG. 56, deployment of the device 120 includes delivering the guide sheath 1402 and the steerable catheter 1404 into an internal chamber (e.g., left ventricle LV) of the heart H. The steerable catheter 1404 is arranged such that the distal end 1454 of the steerable catheter 1404 is adjacent the heart wall W.

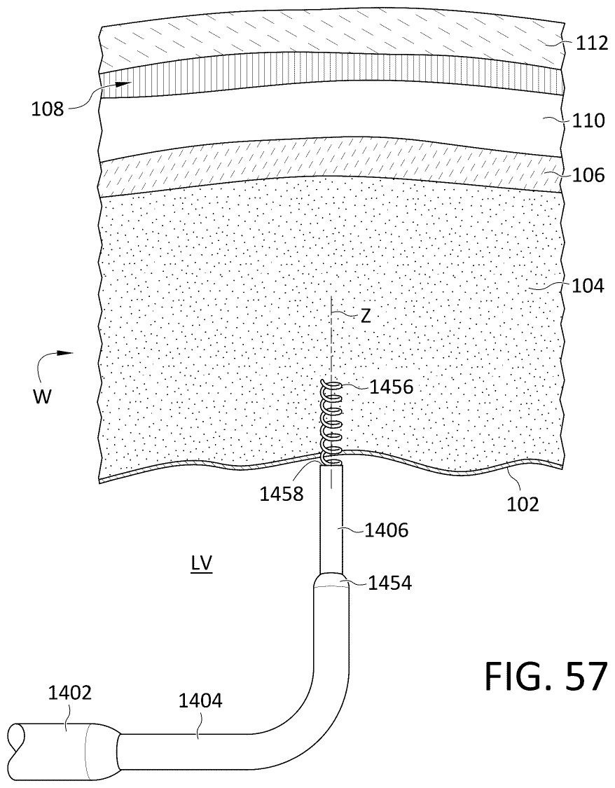

Referring to FIG. 57, the delivery catheter 1406 is extended from the distal end 1454 of the steerable catheter 1404. The anchoring device 1456 of the delivery catheter 1406 is attached to the heart wall W, such as for example by rotating the delivery catheter 1406 about axis Z relative to the steerable catheter 1404 to screw the anchoring device 1456 into the heart wall (i.e., through the endocardium 102 and into the myocardium 104). In some implementations, the distal end 1458 of the delivery catheter 1406 abuts, or is adjacent, the endocardium 102.

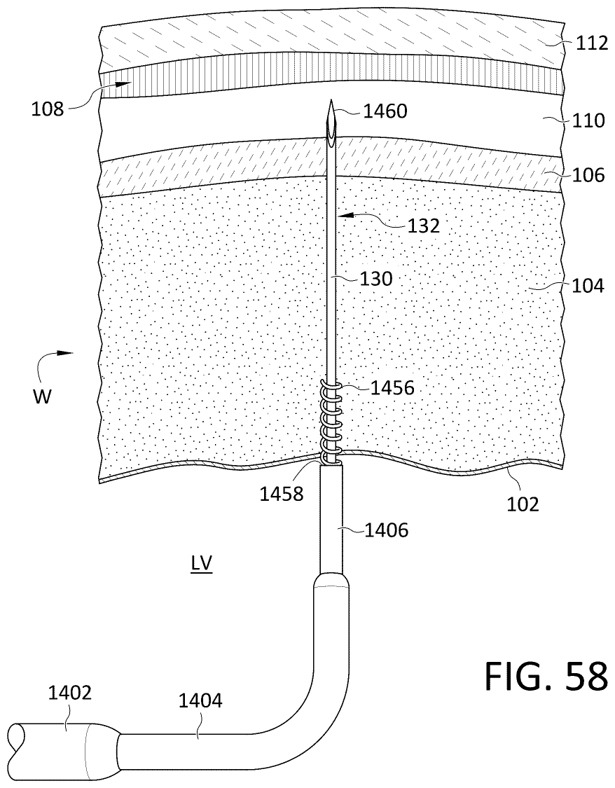

Referring to FIG. 58, the piercing device 130 is delivered into an internal chamber (e.g., left ventricle LV) of the heart H via the delivery catheter 1406. The piercing device 130 can be extended from the distal end 1458 of the delivery catheter 1406 such that the distal end 1460 of the piercing device 130 is extended into the heart wall W. In some implementations, the distal end 1460 of the piercing device 130 is extended through the endocardium 102, the myocardium 104, the epicardium 106, and into the pericardial cavity 110 to form the passage 132. Since the delivery catheter 1406 is anchored to the heart wall W, the location for insertion of the piercing device 130 can be precisely controlled.

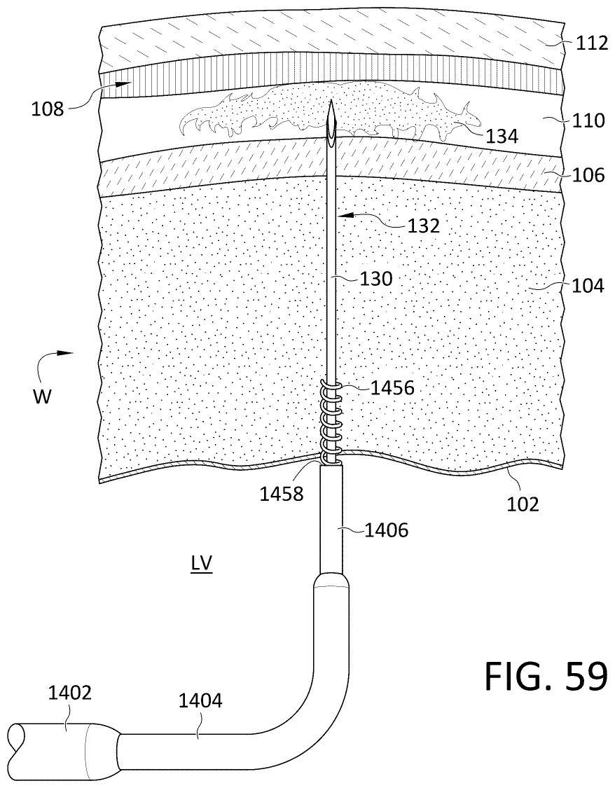

To verify that the piercing device 130 is properly positioned for deploying the anchor 122 into the pericardial cavity 110, a dye 134, or other detectable fluid, can be delivered through the piercing device 130 and injected into the pericardial cavity 110, as shown in FIG. 59. The dye 134 can be detected by any suitable technique such as X-ray, to verify that the piercing device 130 is properly positioned.

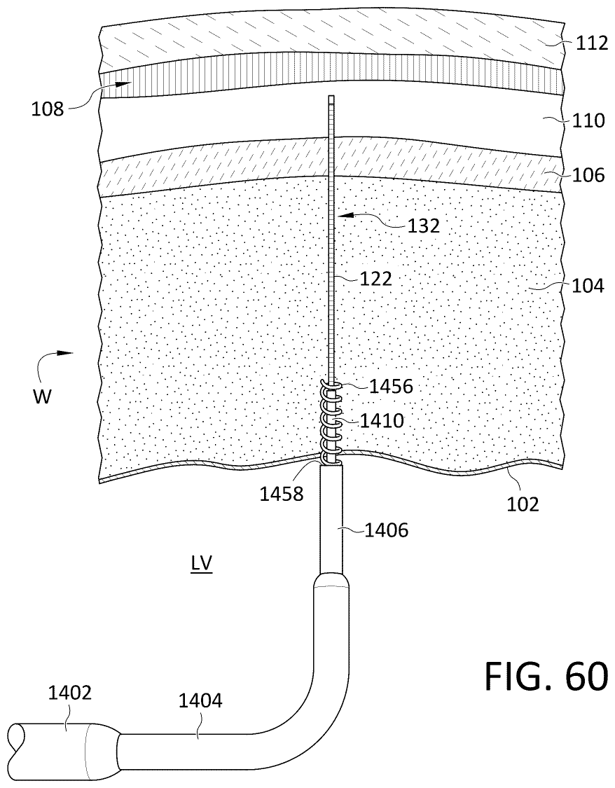

In FIG. 60, the anchor 122 and the hemostatic plug 1410 are extended from the delivery catheter 1406 over the piercing device 130 (FIG. 59) and through the passage 132. As shown in FIG. 60, the anchor 122 remains in the elongated state while sliding along the piercing device 130 into the pericardial cavity 110.

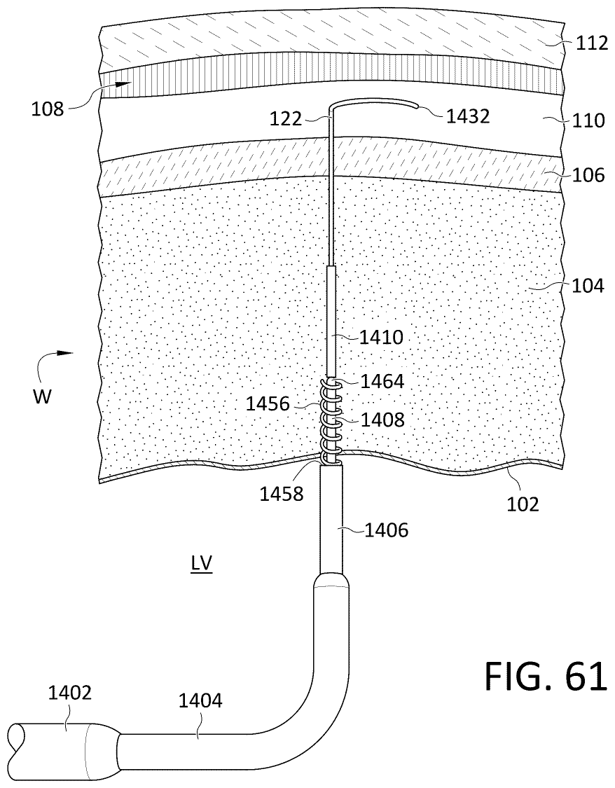

In FIG. 61, the anchor 122 is partially deployed beyond the distal end 1460 (FIG. 58) of the piercing device 130 (FIG. 45). The delivery catheter 1406 is illustrated partially extending from the steerable catheter 1404 and the pusher 1408 is illustrated partially extending from the delivery catheter 1406 through the anchoring device 1456. As the pusher 1408 extends from the delivery catheter 1406, the distal end 1464 of the pusher 1408 engages the hemostatic plug 1410 to push both the hemostatic plug 1410 and the anchor 122 over the piercing device 130 (FIG. 45). As the distal end 1432 of the anchor 122 moves beyond the distal end 1460 of the piercing device 130 (FIG. 45), the anchor 122 can begin to reshape into its curved deployed state. For example, the anchor 122 can include a shape memory alloy that is shape set to the curved deployed state. Thus, the portion of the anchor 122 that is no longer received on the piercing device 130 can revert to the curved deployed state that that shape memory alloy was set to.

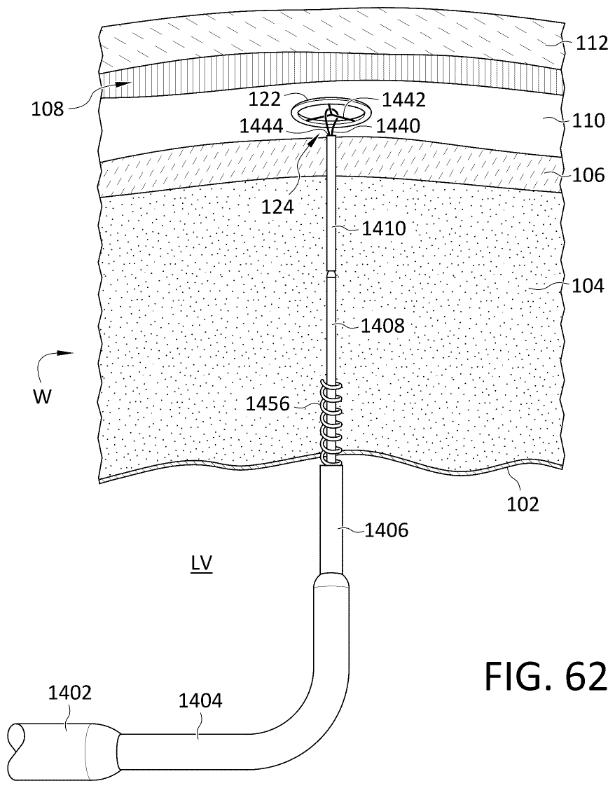

In FIG. 62, the anchor 122 is in the deployed state and the hemostatic plug 1410 is positioned within the passage 132 to prevent bleeding from the passage 132. In some implementations, the distal end 1444 of the hemostatic plug 1410 is at or near the inner wall of the pericardial cavity 110. To reshape the anchor 122, the line 124 is withdrawn by pulling the line 124 in a direction away from the anchor 1422 and into the hemostatic plug 1410. Since the loop 1440 (FIG. 41) of the line 124 passes through the loops 1442 on the anchor 122, pulling the line 124 closes the loop 1440, pulls the hemostatic plug 1410 and the anchor 122 together, and pulls the loops 1442 together to help facilitate, along with any shape setting properties of the anchor 122, reshaping the anchor 122 from the elongated state to the curved deployed state shown in FIG. 62.

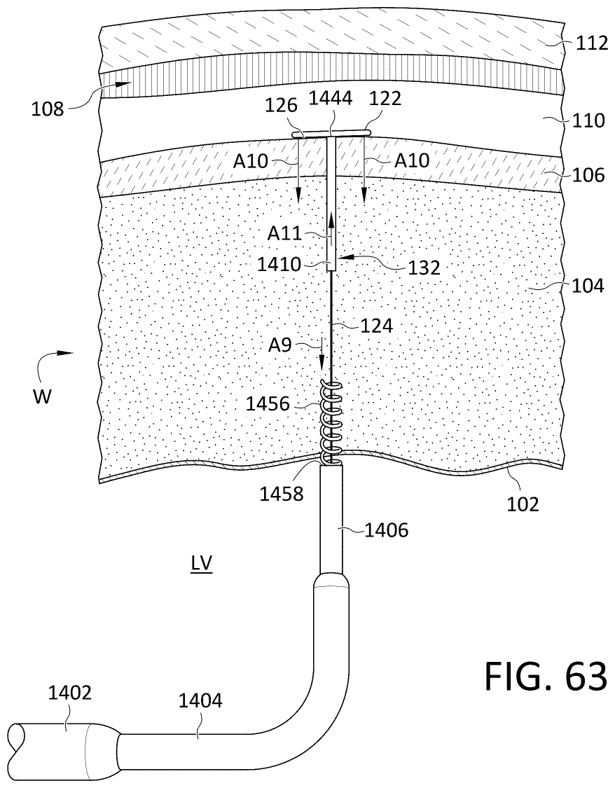

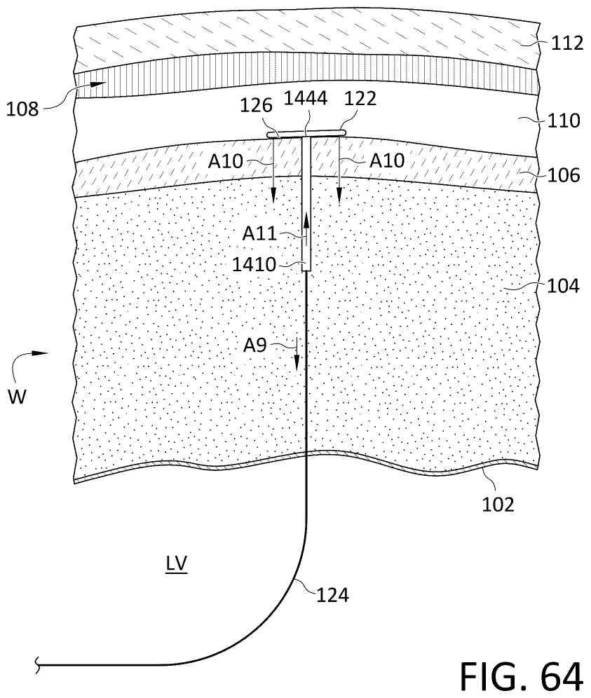

As shown in FIGS. 63-64, the pusher 1408 and piercing device can be removed by withdrawing them from the passage 132 into the delivery catheter 1406. The anchor 122, hemostatic plug 1410, and line 124 are left deployed in the heart wall W with the anchor 122 within the pericardial cavity 110 and the line 124 extending through the epicardium 106, the myocardium 104, and the endocardium 102.

To seat the anchor 122, the delivery catheter 1406 can remain attached to the heart wall W and the distal end 1458 can be pushed against the heart wall W. At the same time, the line 124 can be placed in tension by pulling the line 124 in an inward direction toward the heart chamber as shown by the arrow A9. As a result, the anchor 122 will be pulled against the outward facing surface 126 that partially defines the pericardial cavity 110, as shown by arrows A10. As the line 124 is being pulled in the direction of A9, the hemostatic plug 1410 is urged in the opposite direction, as shown by arrow A11, such that the distal end 1444 of the hemostatic plug 1410 is positioned at or adjacent the center of the anchor 122. The anchor 122 in its deployed state is too large to fit through the passage 132 formed by the piercing device 130. Thus, once the delivery catheter 1406 is removed, further tensioning of the line 124 can pull the heart wall W inward toward the heart chamber (e.g., the left ventricle LV).

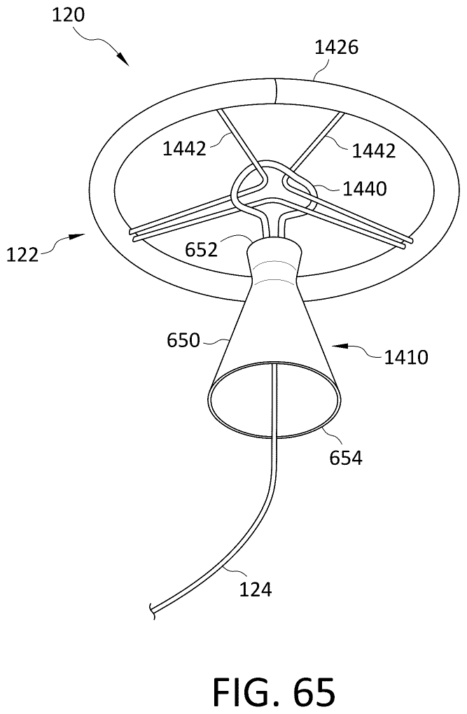

As set forth herein, various examples of the device 120 include a hemostatic plug or tube 1410. The device 120 and the hemostatic plug or tube 1410 can take any of the forms disclosed herein and/or international application publication number WO 2020/219281. Although described in the implementations above as being in the form of a tube, the hemostatic plug 1410 can have a variety of configurations, such as those illustrated in FIGS. 65-82. As will be described, the hemostatic plug 1410 of FIGS. 65-82 in general includes a hemostatic coil and at least one layer of a pliable material extending coaxially around the hemostatic coil. According in some implementations, when positioned within a puncture channel, the pliable material fills a gap between an exterior surface of the hemostatic coil and an inner surface of the puncture channel, reducing or even preventing the flow of blood from the interior of the heart through the puncture channel.

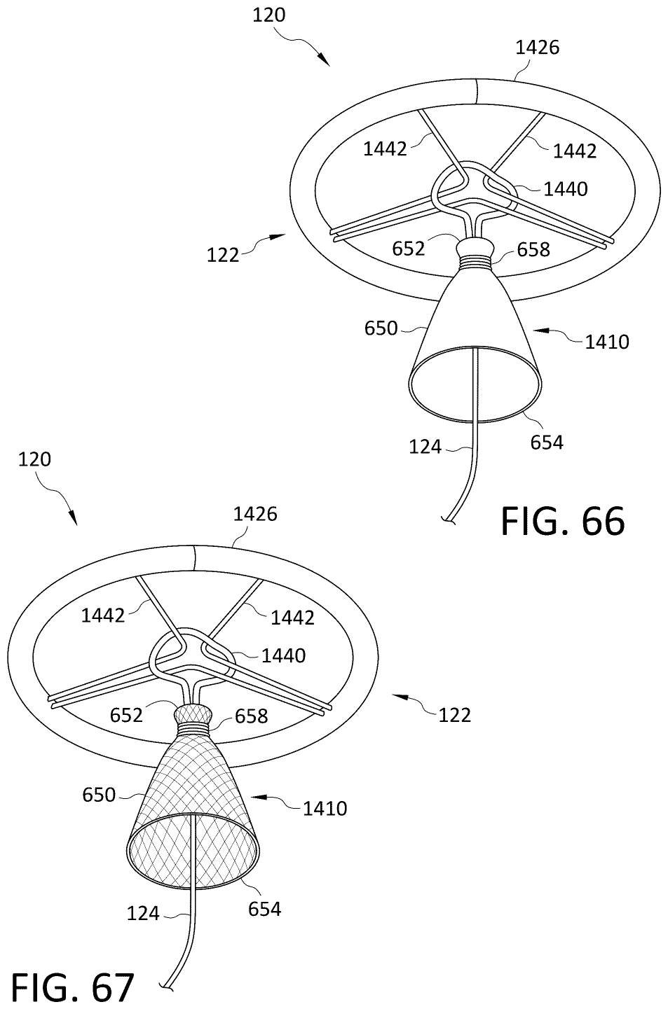

As shown in FIGS. 65 and 66, in some implementations, the hemostatic plug 1410 comprises a skirt 650. The skirt 650 can be formed from, for example, a cloth, PET, or nitinol braid, or another woven or similarly pliable material, such as a mesh or woven fabric, silicone, or polymer. The skirt 650 encircles the line 124 such that the line 124 extends coaxially through a central aperture extending between a first end 652 and a second end 654 of the skirt 650. In some implementations, the first end 652 of the skirt 650 forms an opening that is smaller in size than an opening formed by the second end 654 of the skirt 650. Accordingly, in some implementations, the central aperture of the skirt 650 is tapered in the longitudinal direction.

The skirt 650 has a length extending from the first end 652 to the second end 654 of from about 2 mm to about 25 mm, from about 2 mm to about 15 mm, from about 2 mm to about 10 mm, or any range or sub-range therein. In some implementations, the skirt 650 has a length that is greater than a length of an optional hemostatic coil and/or less than the thickness of the ventricular wall. Although different lengths are possible and contemplated, the length should be long enough to reduce the likelihood that the entire length of the skirt 650 will pass through the surface 126 (FIG. 63) and exit the passage 132 during positioning of device 120, which can result in interference, bunching, or flipping of the skirt 650 during attempts to pull the skirt 650 back into the passage 132 (See FIG. 63).

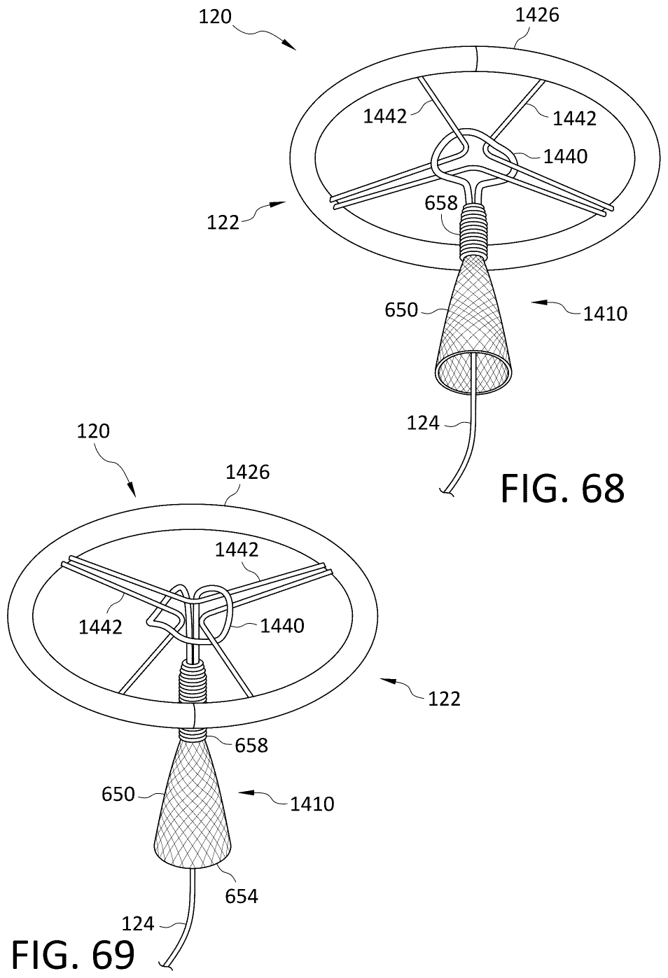

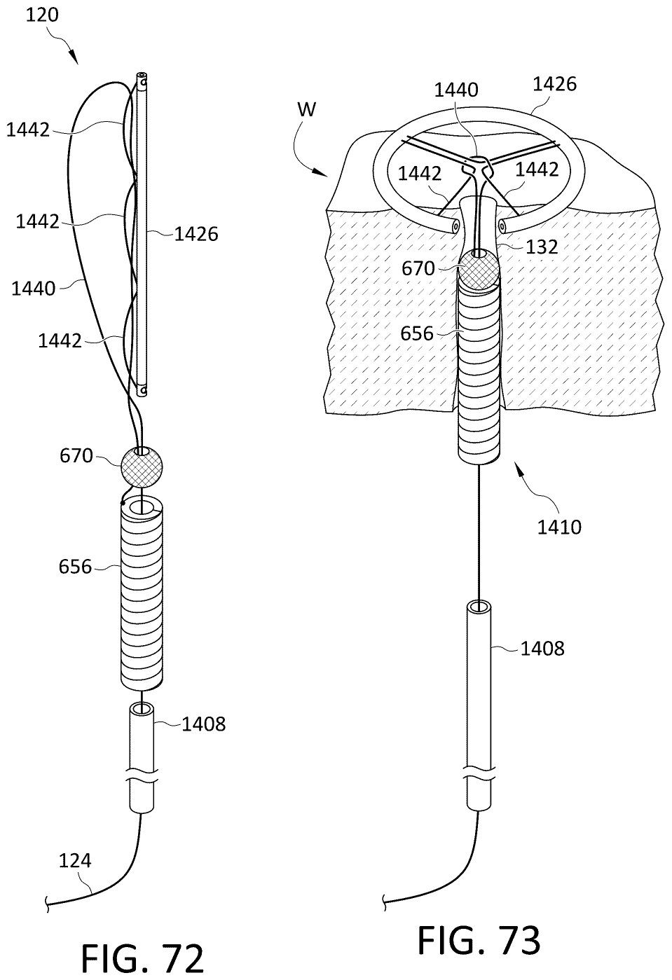

In some implementations, the skirt 650 is positioned over an optional hemostatic tube or coil 656 (not shown in FIG. 65 but see FIG. 72). The hemostatic coil can take a wide variety of different forms. In the examples before FIG. 65, the hemostatic plug or tube 1410 includes only one component (i.e., the tube). In some implementations illustrated by FIGS. 65-82 the hemostatic plug or tube can include more than one component, such as the skirt 650, the hemostatic tube or coil 656, etc. The optional hemostatic tube or coil 656 can be the same as any of the hemostatic plugs or tubes disclosed herein and/or in international application publication number WO 2020/21928, smaller versions of the hemostatic plugs or tubes disclosed herein and/or in international application publication number WO 2020/21928, a tube made by winding or coiling a wire around a mandrel, a tube with a continuous or discontinuous helical or spiral cut, etc. Although obscured (or not included) in the FIG. 65 example, by the skirt 650, the hemostatic coil 656 (if included) extends coaxially within the central aperture of the skirt 650 and is positioned at least partially between an interior surface of the skirt 650 and the line 124. Accordingly, as the skirt 650 is compressed inward by the passage 132 (See FIG. 63), the line 124 remains movable through the hemostatic coil 656, and, therefore, the skirt 650, as may be required to properly seat and secure the anchor 122. In the example shown in FIG. 65, the hemostatic coil 656 (if included) is positioned substantially within the skirt 650, such that a distal end of the hemostatic coil 656 does not substantially extend from the first end 652 of the skirt 650. However, in other implementations, the skirt 650 is offset from the distal end of the hemostatic coil 656 (see, e.g., FIGS. 68-69).

The optional hemostatic coil 656 has a length extending from a distal end to a proximal end of from about 2 mm to about 25 mm, from about 2 mm to about 15 mm, or from about 2 mm to about 15 mm. Although different lengths are possible and contemplated, it is believed that the length of the hemostatic coil 656 should be limited so as not to prevent the device 120 from being cinched and locked to another device, as described herein with reference to FIGS. 32-37, for example.

In some implementations, the skirt 650 is cinched at or near the first end 652 of the skirt 650 (FIG. 66). The skirt 650 can be cinched, for example, using a wrap 658, such as a wrap formed by winding a thread or other material around the outer surface of the skirt 650 to reduce the size of the opening of the first end 652 or a diameter of the central aperture of the skirt 650 near the first end 652. As another example, the skirt 650 can be cinched by inserting sutures through the skirt 650 at or near the first end 652 and pulling the sutures to draw the skirt 650 inward, thereby reducing the size of the central aperture of the skirt 650. In other implementations, such as the example depicted in FIG. 65, the skirt 650 is not cinched.

When the anchor 122 is deployed, the optional hemostatic tube and the skirt 650 form a plug in the passage (e.g., passage 132 in FIG. 63). As the line 124 is pulled away from the anchor 122, the hemostatic tube (if included) and the skirt 650 are pulled toward the anchor 122 are pulled together. As the line 124 is further manipulated to seat the anchor 122, the skirt 650 opens, filling the space between the outer diameter of the hemostatic tube and the interior surface of the passage 132 or to fill the space between the line 124 and the passage 132 if a hemostatic tube is not included. Accordingly, the pliable skirt 650 is effective to fill space in the passage 132 to prevent blood leak.

Turning now to FIG. 67, an alternative example of a hemostatic plug 1410 is illustrated. In FIG. 67, the hemostatic plug 1410 is in the form of a skirt 650 formed from nitinol and PET woven together. The skirt 650 is expandable and compressible, thereby enabling the skirt 650 to expand or be compressed within the passage 132 through the muscle of the heart to fill the passage 132. In some implementations, the skirt 650 is used in conjunction with the hemostatic tube (e.g., as described above), or can be used alone as the hemostatic plug 1410 without the hemostatic coil.

The skirt 650 encircles the line 124 such that the line 124 extends coaxially through a central aperture extending between a first end 652 and a second end 654 of the skirt 650. In some implementations, the central aperture of the skirt 650 is tapered in the longitudinal direction, although in other implementations, the opening at the first end 652 and the opening at the second end 654 are substantially the same size.

In some implementations, the skirt 650 is positioned over an optional hemostatic coil 656 (not shown in FIG. 67 but see FIG. 72). Although obscured (if included) in FIG. 67 by the skirt 650, the hemostatic coil 656 extends coaxially within the central aperture of the skirt 650 and is positioned at least partially between an interior surface of the skirt 650 and the line 124. Accordingly, as the skirt 650 is compressed inward by the passage 132 (See FIG. 63), the line 124 remains movable through the skirt 650, as may be required to properly seat and secure the anchor 122. In the example shown in FIG. 67, the hemostatic coil 656 (if included) is positioned substantially within the skirt 650, such that a distal end of the hemostatic coil 656 does not substantially extend from the first end 652 of the skirt 650. However, in other implementations, the skirt 650 is offset from the distal end of the hemostatic coil 656 (not shown).

In some implementations, the skirt 650 is cinched at or near the first end 652 of the skirt 650. The skirt 650 can be cinched, for example, using a wrap 658, such as a wrap formed by winding a thread or other material around the outer surface of the skirt 650 to reduce the size of the opening of the first end 652 or a diameter of the central aperture of the skirt 650 near the first end 652. As another example, the skirt 650 can be cinched by weaving sutures through the skirt 650 at or near the first end 652 and pulling the sutures to draw the skirt 650 inward, or by weaving the nitinol and PET tighter at the first end 652 than at the second end 654, thereby reducing the size of the central aperture of the skirt 650. In other implementations, the skirt 650 is not cinched.

In some implementations including a skirt 650, the skirt 650 is positioned within the passage 132 (See FIG. 63) and expands to fill the passage 132. For example, the self-expanding material of the skirt 650 can expand to fill the passage 132, such as the space between the outer surface of the hemostatic coil 656 and the passage 132 when the hemostatic coil 656 is present. According to some implementations, the skirt 650 is collapsible, such as in response to pressure from the contraction of the wall, thereby reducing the central aperture of the skirt 650 and reducing or blocking the flow of blood through the passage 132.

As described herein, in some implementations, the skirt 650 is offset from the distal end of the hemostatic coil 656. Such a configuration can, in some implementations, reduce the puncture force of the hemostatic plug 1410 as compared to configurations in which the skirt 650 and the hemostatic coil 656 are not offset relative to one another. As used herein, the “puncture force” refers to an amount of force required to advance a needle loaded with a device including the hemostatic plug and the skirt through the myocardium of the heart.

Referring now to FIGS. 68 and 69, a device 120 including a skirt 650 is illustrated. The skirt 650 is offset from the distal end of the hemostatic coil (obscured by the wrap 658 in FIGS. 68 and 69) such that the skirt 650 is separated from the body 1426 by at least a portion of the length of the hemostatic coil. In FIGS. 68 and 69, pulling the line 124 to the loop 1440 pulls the hemostatic plug 1410 and the anchor 122 together. As can be seen by comparing FIGS. 68 and 69 to one another, as the loop 1440 is pulled tighter, the skirt 650 remains spaced apart from the body 1426 by the hemostatic coil.

As shown in FIGS. 68 and 69, a wrap 658 can cover the hemostatic coil in some implementations. The wrap 658 can additionally secure the skirt 650 to the hemostatic coil and enable a gradual dilation from the outer circumference of the hemostatic coil to an outer circumference of the skirt 650. Alternatively, in some implementations, a marker band can be crimped about the outer surface of the skirt 650 to secure the skirt 650 to the hemostatic coil. Marker bands can be made of any radiopaque material or other material that enable the marker band to be visualized during implantation of the device 120. It should be appreciated that marker bands can be positioned elsewhere along the device 120, such as at a proximal end of the skirt, or a distal end of the hemostatic coil to enable visualization of a target zone to be maintained within the wall W of the heart.

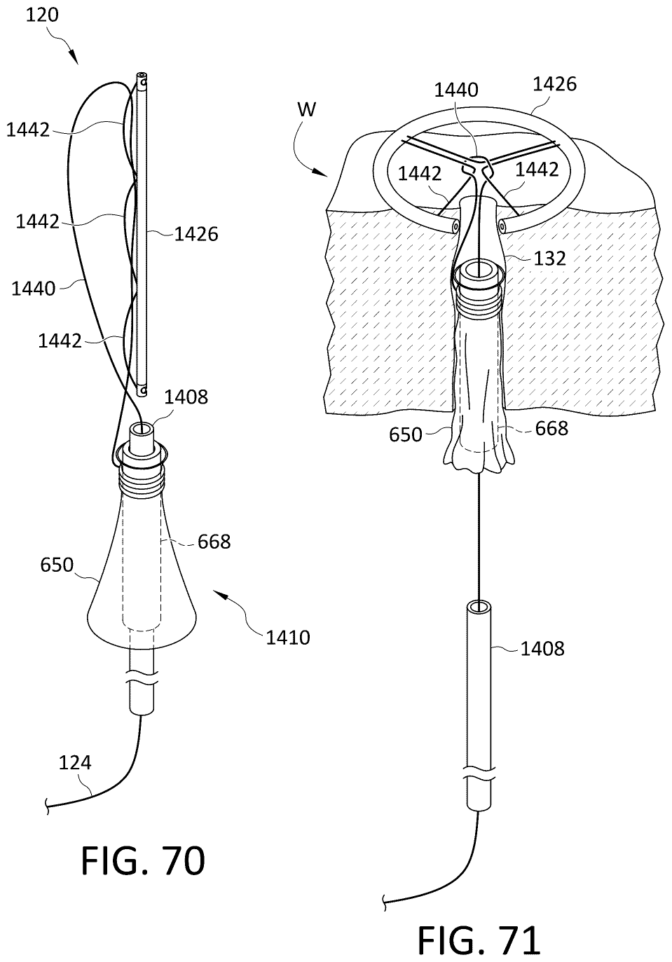

In some implementations, the hemostatic coil is replaced with a sleeve 668. The sleeve 668 is made of a soft flexible material, such as a cloth, PET, or nitinol braid, or another woven or similarly pliable material, such as a mesh or woven fabric, silicone, or polymer, as shown in FIGS. 70 and 71. In such implementations, the sleeve 668 and the skirt 650 can be made from the same material, or a different material. When the sleeve 668 and the skirt 650 are made from the same material, the sleeve 668 and the skirt 650 can be formed from the same piece of material, such as a piece of material folded back on itself, or from separate pieces of material.

FIG. 70 illustrates the body 1426 in an elongated configuration, with the loop 1440 extending through the loops 1442 coupled to the body 1426. A first end of the line 124 forming the loop 1440 is fixed with respect to the skirt 650, and a second end of the line 124 extends through the skirt 650 and the sleeve 668. In FIG. 70, the skirt 650 and the sleeve 668 are positioned over the pusher 1408 for positioning, with the sleeve 668 positioned between the skirt 650 and the pusher 1408. When deployed, as shown in FIG. 71, the line 124 is withdrawn by pulling the line 124 in a direction away from the anchor 1422 and into the hemostatic plug 1410 (the sleeve 668 and the skirt 650). Since the loop 1440 (FIG. 70) of the line 124 passes through the loops 1442 on the anchor 1422, pulling the line 124 closes the loop 1440, pulls the hemostatic plug 1410 and the anchor 1422 together, and pulls the loops 1442 together to help facilitate, along with any shape setting properties of the anchor 1422, reshaping the anchor 1422 from the elongated state to the curved deployed state shown in FIG. 71. As the anchor 1422 is further seated into position, the skirt 650 and the sleeve 668 move within the central passage, filling the space between the wall W and the line 124 when the pusher 1408 is removed.

In still other implementations, the hemostatic plug 1410 includes a hemostatic coil 656 and a fabric bead 670, as shown in FIGS. 72 and 73. In the example in the figures, the line 124 is affixed to the distal end of the hemostatic coil 656, extends through the fabric bead 670, forms the loop 1440, extends back through the fabric bead 670 and through the hemostatic coil 656. Accordingly, the fabric bead 670 is positioned adjacent the distal end of the hemostatic coil 656, between the loop 1440 and the hemostatic coil 656. The fabric bead 670 can be formed from any suitable material, such as a cloth, PET, or nitinol braid, or another woven or similarly pliable material, such as a mesh or woven fabric, silicone, or polymer.

FIG. 72 illustrates the body 1426 in an elongated configuration, with the loop 1440 extending through the loops 1442 coupled to the body 1426. A first end of the line 124 forming the loop 1440 is fixed with respect to the hemostatic coil 656, and a second end of the line 124 extends through the fabric bead 670 and the hemostatic coil 656. In FIG. 72, the hemostatic coil 656 and the fabric bead 670 are positioned over the pusher 1408 for positioning, with the hemostatic coil 656 positioned between the fabric bead 670 and the pusher 1408. When deployed, as shown in FIG. 73, the line 124 is withdrawn by pulling the line 124 in a direction away from the anchor 1422 and into the hemostatic plug 1410 (the fabric bead 670 and the hemostatic coil 656). Since the loop 1440 (FIG. 72) of the line 124 passes through the loops 1442 on the anchor 1422, pulling the line 124 closes the loop 1440, pulls the hemostatic plug 1410 and the anchor 1422 together, and pulls the loops 1442 together to help facilitate, along with any shape setting properties of the anchor 1422, reshaping the anchor 1422 from the elongated state to the curved deployed state shown in

FIG. 73. As the anchor 1422 is further seated into position, the hemostatic coil 656 and the fabric bead 670 move within the central passage, filling the space between the wall W and the line 124 when the pusher 1408 is removed.

Although depicted in FIG. 73 as being positioned within the central passage, in some implementations, the fabric bead 670 has a width (e.g., diameter) that is larger than a width of the central passage such that pulling the line 124 closes the loop 1440 and pulls the hemostatic plug 1410 and the anchor 1422 together, but does not pull the fabric bead 670 into the central passage. Instead, the fabric bead 670 is pulled into contact with the end of the central passage and flexes or bunches as the line 124 is further pulled. Accordingly, in such implementations, the fabric bead 670 works as a pledget and plug for the epicardial side of the central passage, blocking the flow of fluid through the central passage.

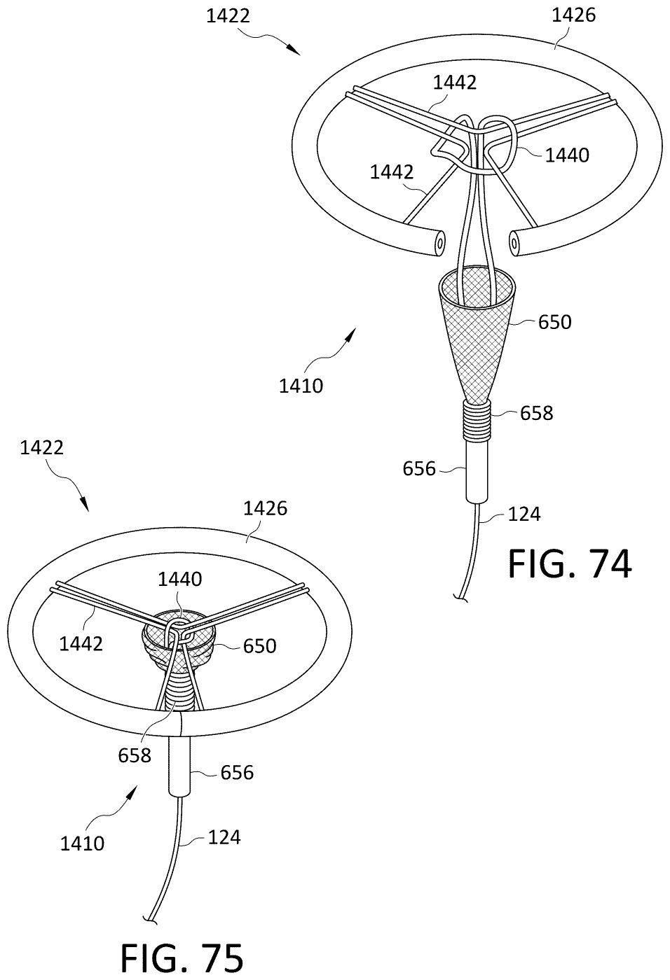

In still further implementations, such as the examples shown in FIGS. 74 and 75, the skirt 650 is positioned on the distal end of the hemostatic coil 656 and between the hemostatic coil 656 and the anchor 1422. In the example shown in FIG. 74, the skirt 650 is tapered along a length of the skirt 650, and has a width that varies. The shape of the skirt 650 can be imparted, for example, through the use of a shape memory material, stitching or cinching the material of the skirt to define a waist, or the like. Although the particular shape of the skirt 650 can vary depending on the particular implementations, in some implementations, the skirt 650 is shaped to enable the skirt to expand laterally when it is compressed in the longitudinal direction, as shown in FIG. 75.

In FIGS. 74 and 75, the skirt 650 is coupled to the distal end of the hemostatic coil 656 and can be secured in place by a wrap 658, such as a wrap formed by winding a thread or other material around the outer surface of the skirt 650 and the hemostatic coil 656. As in other implementations shown and described herein, the wrap 658 can extend along any desired length of the hemostatic coil 656, such that the wrap 658 can cover substantially all of the length of the hemostatic coil 656 or a portion of the length of the hemostatic coil 656.

In use, as the line 124 is pulled, the loop 1440 is closed and the hemostatic plug 1410 and the anchor 1422 are pulled together. The skirt 650 is longitudinally compressed between the anchor 1422 and the hemostatic coil 656, which causes the skirt 650 to expand laterally, as shown in FIG. 75. The lateral expansion of the skirt 650, in some implementations, is sufficient to prevent the skirt 650 from being pulled into the central passage (not shown in FIG. 75). Accordingly, the skirt 650 becomes compressed between the end of the central passage and the anchor 1422 where it works as a pledget and plug for the epicardial side of the central passage, blocking the flow of fluid through the central passage.

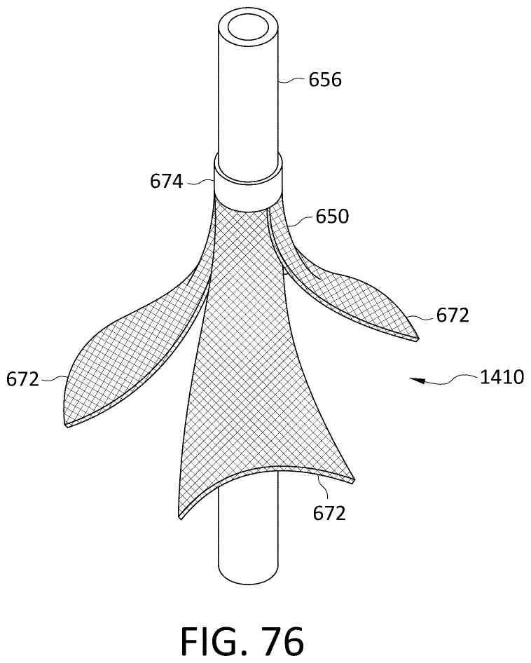

Turning now to FIG. 76, another example of a hemostatic plug 1410 is shown. Although not depicted in FIG. 76, it should be appreciated that the hemostatic plug 1410 can be used with any of the anchor devices shown and described herein. The hemostatic plug 1410 in FIG. 76 includes a hemostatic coil 656 and a skirt 650. The skirt 650 includes a plurality of petals 672 joined to a collar 674. Although three petals 672 are illustrated in FIG. 76, it is contemplated that any number of petals can be included, depending on the particular implementation. For example, the skirt 650 can include two, three, four, or even five or more petals 672. The petals 672 can have a tapered shape such that an end of the petal 672 proximate the collar 674 (e.g., the proximal end) has a width that is less than a width of a free end of the petal 672 (e.g., the distal end). Sides extending from the proximal end to the distal end can be straight or curved. Additionally, a free edge of each petal 672 can be straight or curved (as shown in FIG. 76). The petals 672 are made of a soft flexible material, such as a cloth, PET, or another woven or similarly pliable material, such as a mesh or woven fabric, silicone, or polymer. The flexibility and shape of the petals 672 enable the petals 672 to move and engage with the tissue, filling space between the hemostatic coil 656 and the central passage, and thereby preventing the flow of fluids. In some implementations, the petals 672 and the collar 674 are made from a continuous piece of material, although it is contemplated that the petals 672 can be separately formed and coupled to the collar 674, such as through stitches, adhesives, or the like.

The collar 674 is similarly made of a soft flexible material, such as a cloth, PET, or another woven or similarly pliable material, such as a mesh or woven fabric, silicone, or polymer. Other materials are contemplated and possible, provided that the collar 674 is couplable to the petals 672 and the hemostatic coil 656. In some implementations, the collar 674 defines an aperture sized to receive the hemostatic coil 656 such that the hemostatic coil 656 extends coaxially through the collar 674.

As shown in FIG. 76, in some implementations, the collar 674 is offset from the ends of the hemostatic coil 656 to increase the likelihood that the petals 672 coupled to the collar 674 are positioned to enable engagement with the tissue during insertion and seating of the device. However, as with other implementations shown and described herein, the specific amount of offset can vary depending on the particular implementations and may depend, for example, on the length of the hemostatic coil 656, the length of the petals 672, and other features included in the device.

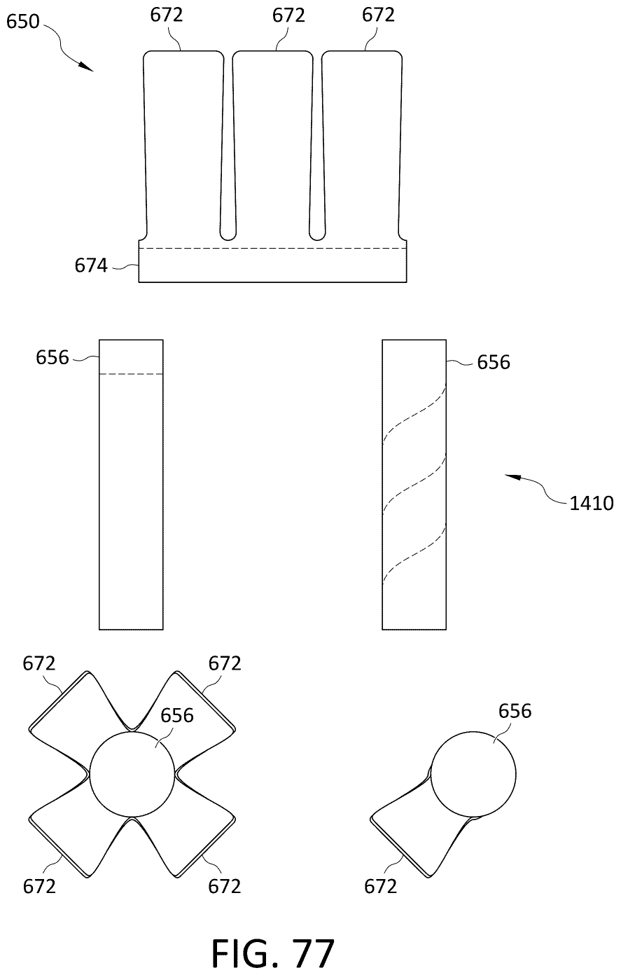

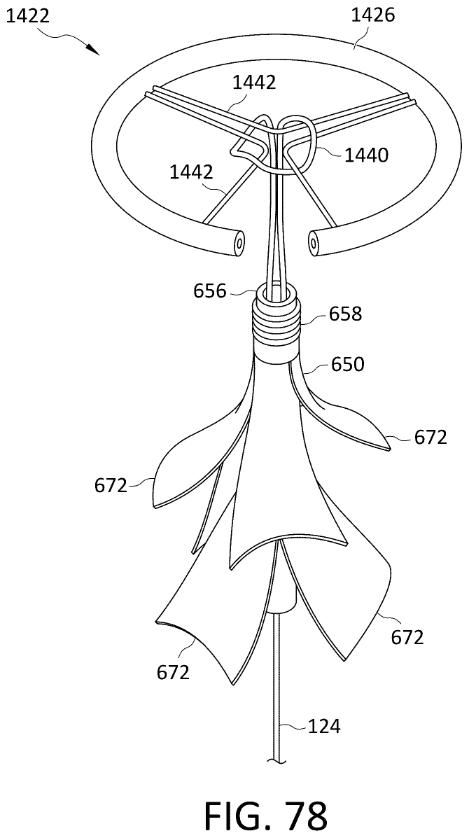

Moreover, in FIG. 76, the skirt 650 is wrapped around the hemostatic coil 656 such that a top edge of the skirt 650 (e.g., an edge of the collar 674) is parallel to an edge of the hemostatic coil 656. Put another way, the skirt 650 is positioned such that an edge of the collar 674 lies in a plane. However, it should be appreciated that, in other implementations, the skirt 650 is wrapped helically around the hemostatic coil 656, as illustrated in FIGS. 77 and 78.

FIG. 77 is a plan view of a skirt 650 including a plurality of petals 672 coupled to a collar 674. FIG. 77 also illustrates a hemostatic coil 656 and a dotted line representing an edge of the collar 674 of the skirt 650. On the left-hand side of FIG. 77, dotted line represents implementations in which the skirt is wrapped circularly around the hemostatic coil 656, as shown in FIG. 76, and the edge of the collar 674 lies in a plane. On the right-hand side of FIG. 77, however, the dotted line is angled with respect to the end of the hemostatic coil 656, representing implementations in which the skirt 650 forms a helix around the hemostatic coil 656, as shown in FIG. 78. Depending on the orientation of the edge of the collar 674, petals 672 extend from the hemostatic coil 656 at the same level (e.g., as shown in the bottom left representation in FIG. 77), or can be layered longitudinally along the hemostatic coil 656 (e.g., as shown in the bottom right representation in FIG. 77 and in FIG. 78).

FIG. 78 illustrates an example in which the skirt 650 is helically positioned around the hemostatic coil 656. In FIG. 78, a wrap 658 secures an end of the skirt 650 to the hemostatic coil 656. The petals 672 extend radially outward in a direction away from the hemostatic coil 656 along the length of the hemostatic coil 656. The petals 672 also extend in a plurality of radial directions, such that the petals 672 extend in at least two different radial directions. In contrast to the example depicted in FIG. 76, the example in FIG. 78 can enable a greater number of petals 672 to be affixed to the collar 674 and to extend in a greater number of radial directions, thereby enabling increased engagement with the tissue in the central passage.

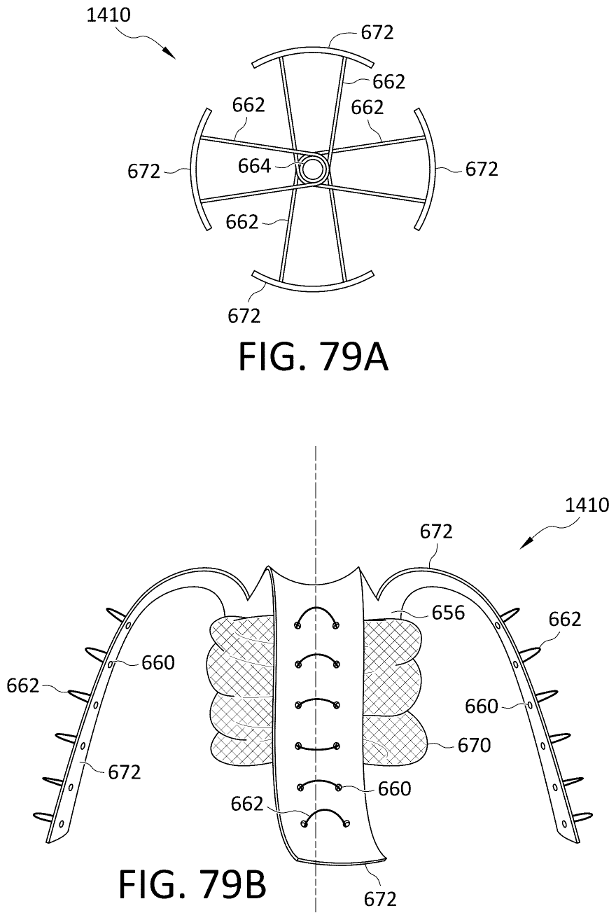

Turning now to FIGS. 79A and 79B, another example of a hemostatic plug is illustrated. FIG. 79A is a top view of a hemostatic plug 1410 having a plurality of petals held together by looping sutures around a needle, while FIG. 79B is a side view of the hemostatic plug 1410 after removal of the needle. In FIGS. 79A and 79B, hemostatic plug 1410 includes a plurality of petals 672 that can be sutured together for insertion into the passage in the heart wall. The petals 672 can be formed, for example, by laser cutting the hemostatic coil or tube 656 along a portion of the length of the hemostatic coil 656. As shown in FIG. 79B, the petals 672 include a plurality of eyelets 660 through which a suture 662 can be threaded to form loops for each petal 672. After the sutures 662 are threaded through the eyelets 660 of each petal 672, a needle 664 (shown in FIG. 79A and represented as a dotted line in FIG. 79B) can be run through loops formed by the suture 662 to hold the petals 672 in place for insertion into the passage in the heart wall. Once inserted into the passage, the needle 664 can be removed from the hemostatic plug 1410 (e.g., by retracting the needle 664 through the remaining length of the hemostatic coil 656) and the petals 672 can be reoriented to fill the gap in the passage.

In the example illustrated in FIG. 79B, the hemostatic plug 1410 also includes a fabric bead 670 positioned about the hemostatic coil 656, although the fabric bead 670 can be omitted or replaced with other features described herein in various implementations.

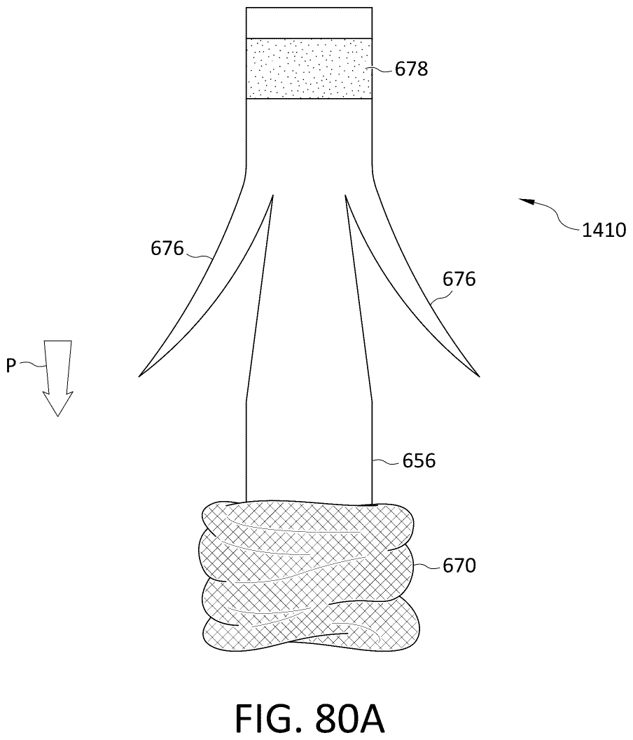

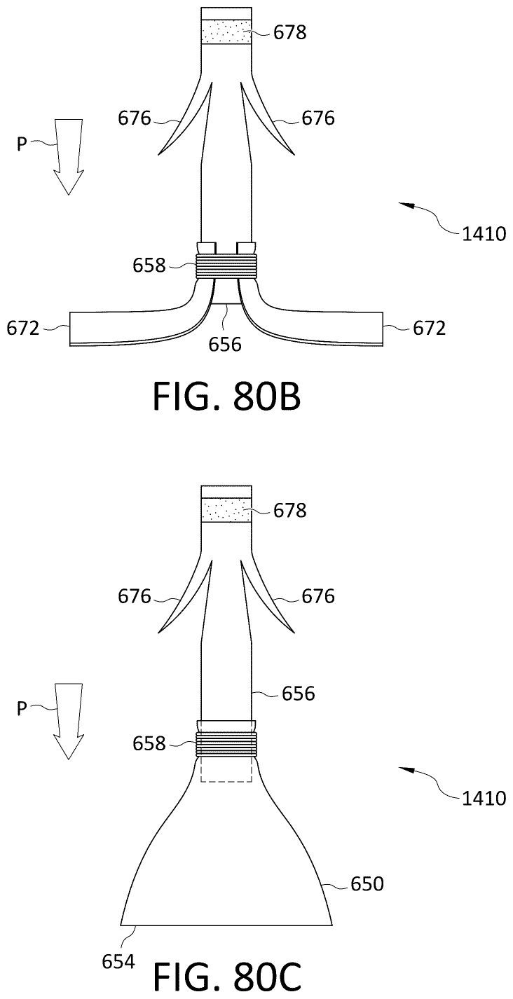

In some implementations, the hemostatic plug 1410 includes one or more optional features. For example, some implementations include one or more marker bands, barbs, or additional collapsible features. Although these features will be described in conjunction with the examples in FIGS. 80A-82, it should be appreciated that one or more of these optional features can be incorporated into any of the implementations shown and described herein in any combination. For example, some implementations can include a marker band and barbs, a marker band and collapsible features, barbs and collapsible features, or the like. Additionally, any one or more of these features can be incorporated into implementations including, but not limited to, skirts, fuzzy beads, petals, or combinations thereof.

Moreover, although the hemostatic plug in the examples in FIGS. 80A-82 is illustrated as a stand-alone device, it is contemplated that the hemostatic plug can be incorporated into an anchor device, as with various hemostatic plugs described above. Accordingly, the hemostatic plug of the various implementations described herein can be used in conjunction with an anchor to close or plug a central passage after the anchor is passed through the central passage, or the hemostatic plug can be used to plug a central passage that was formed but deemed unfit for implant deployment or otherwise unused. Additionally, it should be appreciated that, when used in conjunction with an anchor device (such as the anchor devices described hereinabove), the anchor device and hemostatic plug can be delivered in one step or two steps. For example, the anchor device and hemostatic plug can be joined together for delivery, as described with reference to FIGS. 65-75, or the hemostatic plug can be delivered after the anchor device is delivered and positioned.

FIGS. 80A-80C illustrate various examples in which the hemostatic plug 1410 includes a plurality of barbs 676 and a marker band 678. More particularly, in FIGS. 80A-80C, the hemostatic coil 656 has a plurality of barbs 676 to secure the hemostatic plug 1410 within the central passage of the heart wall W. In some implementations in which the hemostatic coil 656 is formed from a NiTi tube, the tube is laser cut to form the plurality of barbs 676. In other implementations, the plurality of barbs 676 are separately formed and coupled to the hemostatic coil 656.

The plurality of barbs 676 are shaped to enable each barb to resist movement in the proximal direction (e.g., toward the interior of the heart) and to secure the hemostatic plug 1410 in place. The barbs 676 can be tapered along the length of each barb, such that a width of each barb 676 at a point where the barb is affixed to the hemostatic coil 656 is greater than a width of the barb at a free end of the barb. In some implementations, the plurality of barbs 676 are tapered with respect to the hemostatic coil 656 such that each barb extends radially outward in the proximal direction P along the hemostatic coil 656, as illustrated in FIGS. 80A-C. Accordingly, once deployed within the central passage, the plurality of barbs 676 prevent the hemostatic plug 1410 from re-entering the interior of the heart.

The plurality of barbs 676 can be formed from NiTi, as described above, or can be formed from another material having sufficient strength to enable the plurality of barbs 676 to remain in position. In some implementations, the material is moderately flexible such that the barbs 676 can be slightly compressed or maneuvered during positioning, but rigid enough to prevent the plurality of barbs 676 from bending or compressing after deployment.

The hemostatic plug of FIGS. 80A-C optionally further includes a marker band 678 positioned near a distal end of the hemostatic coil 656. The marker band can be made of any radiopaque material or other material that enable the marker band to be visualized during implantation of the hemostatic plug 1410. Accordingly, the marker band 678 can enable a user to properly position the hemostatic plug with respect to the wall W by providing visualization of the distal end of the hemostatic plug 1410. Although the marker band 678 can be positioned at other locations along the length of the hemostatic coil 656, it can be advantageous to enable visualization of the distal end of the hemostatic coil 656 to ensure that the distal end of the hemostatic coil 656 remains within the thickness of the wall W since the plurality of barbs 676 resist movement of the hemostatic plug 1410 in the proximal direction.

It should be appreciated that, although the examples in FIGS. 80A-80C include only one marker band, any number of marker bands can be included. For example, in some implementations, a series or grouping of multiple marker bands can be used, or marker bands can be positioned at different locations along the length of the hemostatic coil 656 (e.g., a single, thick band at a distal end of the hemostatic coil and two, thin bands at a proximal end of the hemostatic coil). Other configurations are contemplated and possible.

In FIG. 80A, the hemostatic plug 1410 includes a fabric bead 670 positioned at the proximal end of the hemostatic coil 656. The fabric bead 670 can be, for example, the fabric bead described above in accordance with FIGS. 72 and 73. The fabric bead 670 can be formed from any suitable material, such as a cloth, PET, or nitinol braid, or another woven or similarly pliable material, such as a mesh or woven fabric or polymer. In the example illustrated in FIG. 80A, the hemostatic coil 656 extends coaxially through the aperture of the fabric bead 670. The fabric bead 670 can be affixed to the outer surface of the hemostatic coil 656, such as through the use of an adhesive or the like.

In other implementations, such as the example depicted in FIG. 80B, a plurality of flexible petals 672 are affixed to the proximal end of the hemostatic coil 656. In FIG. 80B, the petals 672 are secured to the end of the hemostatic coil 656 by a wrap 658 that compresses the petals 672 against the outer surface of the hemostatic coil 656. It should be appreciated that, although the petals 672 in FIG. 80B are depicted as being individual petals 672, in some implementations, the petals 672 can be secured to a collar, as described in conjunction with FIGS. 76-78.

As with the petals described in other implementations herein, the petals 672 in the example in FIG. 80B can be made of a soft flexible material, such as a cloth, PET, or another woven or similarly pliable material, such as a mesh or woven fabric, silicone, or polymer. The petals 672 can have a tapered shape such that an end of the petal 672 proximate the hemostatic coil 656 (e.g., the proximal end) has a width that is less than a width of a free end of the petal 672 (e.g., the distal end). Sides extending from the proximal end to the distal end can be straight or curved. Additionally, a free edge of each petal 672 can be straight or curved.

In still other implementations, the hemostatic plug 1410 includes a skirt 650 affixed to the proximal end of the hemostatic coil 656, as shown in FIG. 80C. In FIG. 80C, the skirt 650 is secured to the hemostatic coil 656 by a wrap 658, although it should be appreciated that the skirt 650 can be secured, affixed, or coupled to the hemostatic coil 656 by any of the variety of methods described herein.

In some implementations, the skirt 650 is formed from, for example, a cloth, PET, or nitinol braid, or another woven or similarly pliable material, such as a mesh or woven fabric or polymer. The skirt 650 has a length extending from the first end 652 to the second end 654 of from about 2 mm to about 25 mm, from about 2 mm to about 15 mm, from about 2 mm to about 10 mm, or any range or sub-range therein. In some implementations, the skirt 650 has a length that is greater than a length of the hemostatic coil and less than the thickness of the ventricular wall. Different lengths are possible and contemplated.

In FIGS. 80A-80C, the feature on the proximal end of the hemostatic coil 656 (e.g., the fabric bead 670, the petals 672, and the skirt 650) function as described in other implementations herein. In general, the feature expands to fill a space between the outer surface of the hemostatic coil 656 and an inner surface within the central passage, thereby reducing or even eliminating the flow of blood through the space.

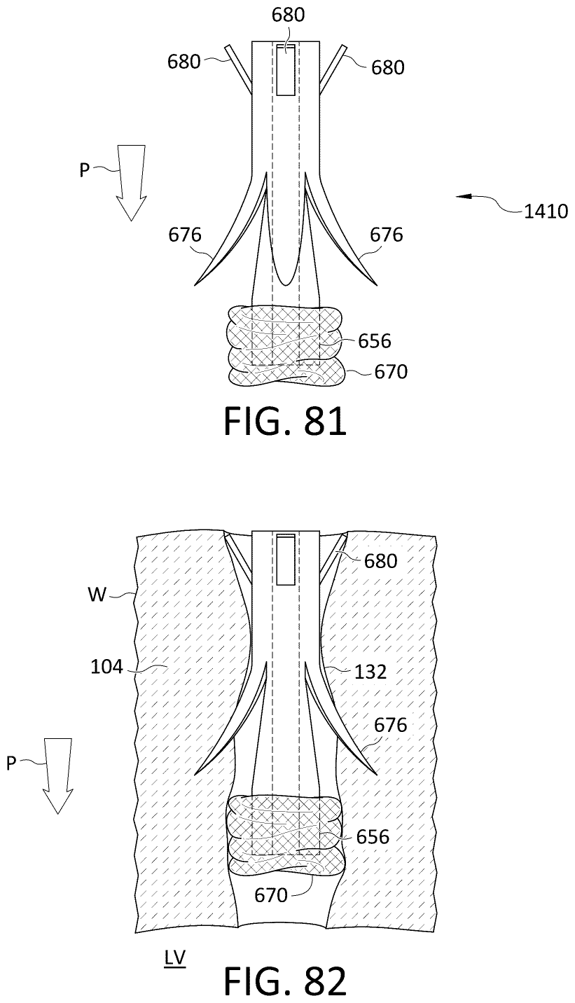

Turning now to FIG. 81, a hemostatic plug 1410 including collapsible features 680 is shown. The hemostatic plug 1410 in FIG. 81 further includes a hemostatic coil 656 having a plurality of barbs 676 and an optional fabric bead 670 positioned on the proximal end of the hemostatic coil 656. The collapsible features 680 extend from the distal end of the hemostatic coil 656.

As illustrated in FIG. 81, the collapsible features 680 are in the form of a plurality of projections, each of which has a free end and a coupled end, with the coupled end being coupled to the hemostatic coil 656. In some implementations, the coupled end of each of the projections can be coupled to one or more other projections as well as the hemostatic coil 656, or each of the projections can be independently coupled to the hemostatic coil 656. Moreover, in some implementations, the coupled end of the projections can be coupled to an intermediary structure (e.g., a collar) that is coupled to the hemostatic coil 656. Accordingly, the projections can be directly or indirectly coupled to the hemostatic coil 656.

The collapsible features 680 are formed from a material that is compressible and/or flexible, such as a cloth, PET, or nitinol braid, or another woven or similarly pliable material, such as a mesh or woven fabric or polymer. The compressibility and/or flexibility of the collapsible features 680 enable the collapsible features 680 to vary in an amount of space occupied by the features. For example, when not in contact with external surfaces, the collapsible features 680 can extend radially outward from the hemostatic coil 656, and in response to pressure being applied by one or more external surfaces, the collapsible features 680 can be compressed or moved radially inward toward the hemostatic coil 656. Accordingly, the collapsible features 680 can be compressed while the hemostatic plug 1410 is moved into position within the wall W, and expand to fill the area within the central passage.

In some implementations, the features 680 can be held radially inward until a wire or needle 664 is removed. For example, the features 680 can be the same or similar to the petals 672 illustrated by FIGS. 79A and 79B or can have any of the features of the petals 672.

In FIG. 82, the hemostatic plug 1410 illustrated in FIG. 81 is depicted in a deployed configuration within a heart wall W. As shown in FIG. 82, the fabric bead 670 and the collapsible features 680 expand radially to fill space at the proximal and distal ends of the hemostatic coil 656, respectively. The barbs 676 also extend radially outward from the hemostatic coil 656, increasing resistance of the hemostatic plug 1410 to move in the proximal direction P toward the interior of the heart (e.g., the left ventricle LV). Accordingly, the hemostatic plug 1410 is operable to plug the central passage (e.g., a puncture channel formed by a needle), obstruct acute bleeding through the central passage, and/or encourage clotting within the central passage, thereby reducing or even preventing chronic bleeding conditions as a result of transcatheter procedures, such as those described herein.

### EXAMPLES

Example 1. A device for sealing a puncture in a human heart, the device comprising:
a hemostatic coil having a distal end and a proximal end; and
at least one layer of a pliable material extending coaxially around the hemostatic coil and configured to reduce an area between the suture line and an inner surface of the puncture channel.

Example 2. The device according to Example 1, wherein the at least one layer forms a skirt having a first end and a second end, wherein the first end of the skirt forms an opening that is smaller in size than an opening formed by the second end of the skirt.

Example 3. The device according to Example 2, wherein a distance between the first end and the second end of the skirt is from about 2 mm to about 25 mm.

Example 4. The device according to Example 2 or 3, wherein the skirt is cinched at or near the first end of the skirt.

Example 5. The device according to Example 4, wherein the skirt is cinched using a wrap formed around an outer surface of the skirt.

Example 6. The device according any of Examples 2-5, wherein the first end of the skirt is offset from the distal end of the hemostatic coil.

Example 7. The device according to Example 6, wherein a wrap extends along an outer surface of the hemostatic coil between the first end of the skirt to the distal end of the hemostatic coil.

Example 8. The device according to any of Examples 2-7, wherein the skirt has a tapered shape.

Example 9. The device according to any of Examples 2-7, wherein the skirt has a variable width between the first end and the second end.

Example 10. The device according to Example 9, wherein the skirt comprises a waist configured to enable the skirt to expand laterally when the skirt is compressed in a longitudinal direction extending from the first end to the second end.

Example 11. The device according to any of Examples 2-10, wherein the skirt comprises a plurality of petals joined to a collar.

Example 12. The device according to Example 11, wherein each of the plurality of petals has a tapered shape such that an end of each of the plurality of petals joined to the collar has a width that is less than a width of a free end of each of the plurality of petals.

Example 13. The device according to Example 11 or 12, wherein the skirt is helically wrapped around the hemostatic coil.

Example 14. The device according to Example 11 or 12, wherein the skirt is wrapped around the hemostatic coil such that an edge of the collar is parallel to an edge of the hemostatic coil.

Example 15. The device according to any of Examples 1-14, further comprising one or more barbs configured to resist movement of the device toward an interior of the human heart when the device is positioned within the puncture channel.

Example 16. The device according to Example 15, wherein the one or more barbs extend from the hemostatic coil.

Example 17. The device according to Example 15 or 16, wherein the one or more barbs comprise nitinol.

Example 18. The device according to any of Examples 2-17, wherein the first end of the skirt is proximate the proximal end of the hemostatic coil.

Example 19. The device according to any of Examples 2-18, wherein the first end of the skirt is proximate the distal end of the hemostatic coil.

Example 20. The device according to any of Examples 2-19, wherein the skirt comprises a weave of nitinol and PET.

Example 21. The device according to any of Examples 2-20, wherein the skirt is positioned over the hemostatic coil such that the hemostatic coil extends coaxially within a central aperture of the skirt.

Example 22. The device according to Example 20 or 21, wherein the nitinol and PET are woven tighter at the first end than at the second end.

Example 23. The device according to any of Examples 20-22, wherein the skirt is cinched at a first end of the skirt using a wrap to reduce a diameter of the central aperture of the skirt at the first end.

Example 24. The device according to any of Examples 1-23, wherein the pliable material comprises a cloth, PET, nitinol braid, woven fabric, mesh fabric, silicone, or woven polymer.

Example 25. The device according to any of Examples 1-24, further comprising a marker band comprising a radiopaque material.

Example 26. The device according to any of Examples 1-25, further comprising an anchoring portion.

Example 27. A device for sealing a puncture in a human heart, the device comprising:
a hemostatic coil having a distal end and a proximal end; and
a fabric bead coupled to the hemostatic coil.

Example 28. The device according to Example 27, wherein the fabric bead is coupled to the proximal end of the hemostatic coil.

Example 29. The device according to Example 27 or 28, further comprising one or more barbs configured to resist movement of the device toward an interior of the human heart when the device is positioned within the puncture channel.

Example 30. The device according to Example 29, wherein the one or more barbs extend from the hemostatic coil.

Example 31. The device according to Example 29 or 30, wherein the one or more barbs comprise nitinol.

Example 32. The device according to any of Examples 27-31, further comprising one or more collapsible features extending from the distal end of the hemostatic coil.

Example 33. The device according to Example 27, wherein the fabric bead is coupled to the distal end of the hemostatic coil.

Example 34. The device according to Example 33, wherein the fabric bead is coupled to the distal end of the hemostatic coil through a line affixed to the distal end of the hemostatic coil.

Example 35. The device according to Example 34, wherein the line extends through the fabric bead in a first direction, forms a loop with an anchor device, and extends through the fabric bead in a second direction.

Example 36. The device according to any of Examples 27-35, wherein the fabric bead comprises a cloth, PET, or nitinol bead.

Example 37. The device according to any of Examples 27-36, further comprising an anchoring portion.

Example 38. A device for sealing a puncture channel in a human heart, the device comprising:
a tissue anchor configured to engage tissue of a heart wall, wherein the wall at least partially defines the internal chamber;
a suture line connected to the tissue anchor; and
at least one layer of a pliable material extending coaxially around the suture line and configured to reduce an area between the suture line and an inner surface of the puncture channel.

Example 39. The device according to Example 38, wherein the at least one layer forms a skirt having a first end and a second end, wherein the first end of the skirt forms an opening that is smaller in size than an opening formed by the second end of the skirt.

Example 40. The device according to Example 39, wherein a distance between the first end and the second end of the skirt is from about 2 mm to about 25 mm.

Example 41. The device according to Example 39 or 40, wherein the skirt is cinched at or near the first end of the skirt.

Example 42. The device according to Example 41, wherein the skirt is cinched using a wrap formed around an outer surface of the skirt.

Example 43. The device according to any of Examples 39-42, wherein the skirt has a tapered shape.

Example 44. The device according to any of Examples 39-42, wherein the skirt has a variable width between the first end and the second end.

Example 45. The device according to Example 44, wherein the skirt comprises a waist configured to enable the skirt to expand laterally when the skirt is compressed in a longitudinal direction extending from the first end to the second end.

Example 46. The device according to any of Examples 39-45, wherein the skirt comprises a plurality of petals joined to a collar.

Example 47. The device according to Example 46, wherein each of the plurality of petals has a tapered shape such that an end of each of the plurality of petals joined to the collar has a width that is less than a width of a free end of each of the plurality of petals.

Example 48. The device according to any of Examples 39-47, wherein the skirt comprises a weave of nitinol and PET.

Example 49. The device according to Example 48, wherein the nitinol and PET are woven tighter at the first end than at the second end.

Example 50. The device according to any of Example 38-49, further comprising a sleeve comprising a flexible material.

Example 51. The device according to Example 50, wherein the sleeve and the at least one layer are formed from the same material.

Example 52. The device according to Example 50 or 51, wherein the sleeve is positioned coaxially between the suture line and the at least one layer.

Example 53. A method for sealing a puncture channel in a human heart having walls that at least partially define an internal chamber, the method comprising:
positioning a distal end of a delivery catheter adjacent an inner surface of a heart wall at a first location; and
delivering a hemostatic plug comprising a hemostatic coil and at least one layer of a pliable material extending coaxially around the hemostatic coil through the catheter to the puncture channel such that the hemostatic plug is positioned within the puncture channel and reduces a flow of blood through the puncture channel.

Example 54. The method according to Example 53, wherein the at least one layer forms a skirt having a first end and a second end, wherein the first end of the skirt forms an opening that is smaller in size than an opening formed by the second end of the skirt.

Example 55. The method according to Example 54, wherein a distance between the first end and the second end of the skirt is from about 2 mm to about 25 mm.

Example 56. The method according to Example 54 or 55, wherein the skirt is cinched at or near the first end of the skirt.

Example 57. The method according to Example 56, wherein the skirt is cinched using a wrap formed around an outer surface of the skirt.

Example 58. The method according to any of Examples 54-57, wherein the first end of the skirt is offset from the distal end of the hemostatic coil.

Example 59. The method according any of Examples 54-58, wherein a wrap extends along an outer surface of the hemostatic coil between the first end of the skirt to the distal end of the hemostatic coil.

Example 60. The method according any of Examples 54-59, wherein the skirt has a tapered shape.

Example 61. The method according to any of Examples 54-59, wherein the skirt has a variable width between the first end and the second end.

Example 62. The method according to Example 61, wherein the skirt comprises a waist configured to enable the skirt to expand laterally when the skirt is compressed in a longitudinal direction extending from the first end to the second end.

Example 63. The method according to any of Examples 54-62, wherein the skirt comprises a plurality of petals joined to a collar.

Example 64. The method according to any of Examples 54-63, wherein the skirt is helically wrapped around the hemostatic coil.

Example 65. The method according to any of Examples 54-64, wherein the skirt comprises a weave of nitinol and PET.

Example 66. The method according to any of Examples 53-65, wherein the hemostatic plug further comprises one or more barbs configured to resist movement of the hemostatic plug toward the internal chamber.

Example 67. The method according to Example 66, wherein the one or more barbs extend from the hemostatic coil.

Example 68. The method according to any of Examples 53-67, further comprising: delivering a tissue anchor through the catheter to the heart wall such that the tissue anchor anchors to the heart wall, wherein the tissue anchor includes a first suture line attached and extends into the internal chamber.

Example 69. The method according to Example 68, wherein the step of delivering the tissue anchor is performed prior to the step of delivering the hemostatic plug.

Example 70. The method according to Example 68, wherein the step of delivering the tissue anchor is performed simultaneously with the step of delivering the hemostatic plug.

Example 71. The method according to any of Examples 68-70, wherein the hemostatic plug is coupled to the tissue anchor through the first suture line.

While various inventive aspects, concepts and features of the disclosures may be described and illustrated herein as embodied in combination in the examples, these various aspects, concepts, and features may be used in many alternative implementations, either individually or in various combinations and sub-combinations thereof. Unless expressly excluded herein all such combinations and sub-combinations are intended to be within the scope of the present application. Still further, while various alternative implementations as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative implementations, whether presently known or later developed. Those skilled in the art may readily adopt one or more of the inventive aspects, concepts, or features into additional implementations and uses within the scope of the present application even if such implementations are not expressly disclosed herein.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of exemplary methods or processes are not limited to inclusion of all steps as being required in all cases, nor is the order that the steps are presented to be construed as required or necessary unless expressly so stated. The words used in the claims have their full ordinary meanings and are not limited in any way by the description of the implementations in the specification.

1. A device for sealing a puncture channel in a human heart, the device comprising:
a hemostatic coil having a distal end and a proximal end; and
at least one layer of a pliable material extending coaxially around the hemostatic coil and configured to be disposed in the puncture channel to reduce an area between a suture line and an inner surface of the puncture channel.

2. The device according to claim 1, wherein the at least one layer of the pliable material forms a skirt having a first end and a second end, wherein the first end of the skirt forms an opening that is smaller in size than an opening formed by the second end of the skirt.

3. The device according to claim 2, wherein the skirt is cinched at or near the first end of the skirt.

4. The device according to claim 3, wherein the first end of the skirt is offset from the distal end of the hemostatic coil.

5. The device according to claim 4, wherein a wrap extends along an outer surface of the hemostatic coil between the first end of the skirt to the distal end of the hemostatic coil.

6. The device according to claim 5, wherein the skirt has a tapered shape.

7. The device according to claim 5, wherein the skirt has a variable width between the first end and the second end.

8. The device according to claim 5, wherein the skirt comprises a plurality of petals joined to a collar, wherein each of the plurality of petals has a tapered shape such that an end of each of the plurality of petals joined to the collar has a width that is less than a width of a free end of each of the plurality of petals.

9. The device according to claim 8, further comprising one or more barbs configured to resist movement of the device toward an interior of the human heart when the device is positioned within the puncture channel.

10. The device according to claim 9, wherein the one or more barbs extend from the hemostatic coil.

11. A method for sealing a puncture channel in a human heart having walls that at least partially define an internal chamber, the method comprising:
positioning a distal end of a delivery catheter adjacent an inner surface of a heart wall at a first location; and
delivering a hemostatic plug comprising a hemostatic coil and at least one layer of a pliable material extending coaxially around the hemostatic coil through the delivery catheter to the puncture channel such that the hemostatic coil and the at least one layer of a pliable material is positioned within the puncture channel and reduce a flow of blood through the puncture channel.

12. The method according to claim 11, wherein the at least one layer of the pliable material forms a skirt having a first end and a second end, wherein the first end of the skirt forms an opening that is smaller in size than an opening formed by the second end of the skirt.

13. The method according to claim 12, wherein the skirt is cinched at or near the first end of the skirt.

14. The method according to claim 13, wherein the first end of the skirt is offset from the distal end of the hemostatic coil.

15. The method according to claim 14, wherein the skirt comprises a waist configured to enable the skirt to expand laterally when the skirt is compressed in a longitudinal direction extending from the first end to the second end.

16. The method according to claim 15, wherein the skirt comprises a plurality of petals joined to a collar.

17. The method according to claim 16, further comprising:
delivering a tissue anchor through the delivery catheter to the heart wall such that the tissue anchor anchors to the heart wall, wherein the tissue anchor includes a first suture line attached and extends into the internal chamber.

18. The method according to claim 17, wherein the step of delivering the tissue anchor is performed prior to the step of delivering the hemostatic plug.

19. The method according to claim 17, wherein the step of delivering the tissue anchor is performed simultaneously with the step of delivering the hemostatic plug.

20. The method according to claim 17, wherein the hemostatic plug is coupled to the tissue anchor through the first suture line.
